(19) United States
(12) Patent Application Publication
(10) Pub. No.: US 2006/0205020 A1
(43) Pub. Date: Sep. 14, 2006

(54) Cell-cycle checkpoint genes

(75) Inventor: Antony Michael Carr, Falmer (GB)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Icos Corporation

(21) Appl. No.: 10/685,258

(22) Filed: Oct. 14, 2003

Related U.S. Application Data
(62) Division of application No. 09/029,047, filed on May 11, 1999, now Pat. No. 6,632,936.

(30) Foreign Application Priority Data
Sep. 6, 1996 (WO) PCT/GB96/09433
Sep. 6, 1995 (GB) 9518220.0

(51) Int. Cl. G01N 33/574; C12Q 1/68; C07H 21/04; C12P 21/06; C07K 14/82

(52) U.S. Cl. 435/7.23; 435/6; 435/69.1; 435/320.1; 435/325; 530/350; 536/23.5; 514/2

(57) ABSTRACT

This invention relates to a class of checkpoint genes and their polypeptide products which control progression through the cell cycle in eukaryotic cells. In particular, this invention relates to Schizosaccharomyces pombe rad3 gene, to its human homologue (ATR), and to their encoded proteins. The invention further relates to assay methods for selecting compounds which modulate the activity of the polypeptide products of these checkpoint genes and the use of the selected compounds in anticancer therapy.

The present invention relates to a class of checkpoint genes which control progression through the cell cycle in eukaryotic cells.

### BACKGROUND TO THE INVENTION

Control of the cell cycle is fundamental to the growth and maintenance of eukaryotic organisms, from yeast to mammals. Eukaryotic cells have evolved control pathways, termed “checkpoints” which ensure that individual steps of the cell cycle are completed before the next step occurs. In response to DNA damage, cell survival is increased both by direct DNA repair mechanisms and by delaying progression through the cell cycle. Depending on the position of the cell within the cycle at the time of irradiation, DNA damage in mammalian cells can prevent (a) passage from G1 into S phase, (b) progression through S phase or (c) passage from G2 into mitosis. Such checkpoints are thought to prevent deleterious events such as replication of damaged DNA and the segregation of fragmented chromosomes during mitosis (Hartwell and Kastan, 1994).

The rad3 gene of Schizosaccharomyces pombe is required for the checkpoints that respond to DNA damage and replication blocks. Rad3 is a member of the lipid kinase subclass of kinases which possess regions having sequence homology to the lipid kinase domain of the p110 subunit of phosphatidylinositol-3 kinase (PI-3 kinase). This subclass also includes the ATM protein defective in ataxia-telangiectasia patients. Cells from ataxia telangiectasia patients (AT cells) have lost the delay to S phase following irradiation and are said to display radio resistant DNA synthesis Painter and Young, 1989). AT cells irradiated in S phase accumulate in G2 with lethal damage, presumably as a consequence of attempting to replicate damaged DNA. AT cells irradiated during G2 display a different phenotype: they do not arrest mitosis after DNA damage and progress through mitosis with damaged DNA (Beamish and Lavin, 1994). Mutations at the A-T locus, to which the ATM gene has been mapped, thus result in disruption of several checkpoints required for an appropriate response to ionising radiation. Other members of this lipid kinase subclass include: Tel1p (Greenwell et al. 1985), a gene involved in maintaining proper telomere length in Saccharomyces cerevisiae; Esr1p: Mec1p and the product of the Drosophila melanogaster mei-41 checkpoint gene (Hari et al. 1995).

### DISCLOSURE OF THE INVENTION

We have analyzed the S. pombe rad3 gene and found that it has a full length amino acid sequence of 2386 amino acids, not the 1070 amino acids described by Seaton et al. 1992. We have determined that this is the direct homologue of S. cerevisiae Esr1p, and that it shares the same overall structure as the ATM gene. The C-terminal region of the rad3 protein contains a lipid kinase domain, which is required for Rad3 function. We have shown that Rad3 is capable of self association. We have also identified a protein kinase activity associated with Rad3.

Further, we have found a human homologue to rad3. This gene, which we have named ATR (ataxia and rad related), displays significantly higher homology to rad3 than it does to the ATM gene.

The human ATR cDNA sequence is set out as Seq. ID No. 1. The amino acid sequence of the ORF from nucleotides 80 and 8011 is set out as Seq. ID. No. 2.

The DNA sequence of the open reading frame (ORF) of rad3 is shown as Seq. ID. No. 3. The 2386 amino acid translation of the gene (nucleotides 585 to 7742 of Seq. ID No. 3) is shown as Seq. ID. No. 4.

Accordingly, in a first aspect, the invention provides the ATR protein of Seq. ID. 2 and homologues thereof, polypeptide fragments thereof, as well as antibodies capable of binding the ATR protein or polypeptide fragments thereof. ATR proteins, homologues and fragments thereof are referred to below as polypeptides of the invention.

In another aspect, the present invention provides a polynucleotide in substantially isolated form capable of hybridising selectively to Seq. ID No 1 or to the complement (i.e. opposite strand) thereof. Also provided are polynucleotides encoding polypeptides of the invention.

Such polynucleotides will be referred to as a polynucleotide of the invention. A polynucleotides of the invention includes DNA of Seq. ID Nos 1 and fragments thereof capable of selectively hybridising to this gene.

In a further aspect, the invention provides recombinant vectors carrying a polynucleotide of the invention, including expression vectors, and methods of growing such vectors in a suitable host cell, for example under conditions in which expression of a protein or polypeptide encoded by a sequence of the invention occurs.

In an additional aspect, the invention provides kits comprising polynucleotides, polypeptides or antibodies of the invention and methods of using such kits in diagnosing the presence of absence of ATR and its homologues, or variants thereof, including deleterious AIR mutants.

The invention further provides assay methods for screening candidate substances for use as compounds for inhibiting or activating ATR activity, or the activity of mutated forms of ATR which are deficient in checkpoint activity. The invention also provides assay methods for screening candidate substances for use as compounds for inhibiting interactions between ATR and other compounds that interact with ATR, including ATR itself.

In a related aspect, the invention also provides a polynucleotide sequence of Seq. ID No. 3 in substantially isolated form, and the protein of Seq. ID No. 4 in substantially isolated form, and novel fragments and variants thereof.

### DETAILED DESCRIPTION OF THE INVENTION

A. Polynucleotides.

Polynucleotides of the invention may comprise DNA or RNA. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or lifespan of polynucleotides of the invention.

Polynucleotides of the invention capable of selectively hybridizing to the DNA of Seq. ID No. 1 will be generally at least 70%, preferably at least 80 or 90% and more preferably at least 95% homologous to the corresponding DNA of Seq. ID No. 1 over a region of a least 20, preferably at least 25 or 30, for instance at least 40, 60 or 100 or more contiguous nucleotides.

It is to be understood that skilled persons may, using routine technique, make nucleotide substitutions that do not affect the polypeptide sequence encoded by the polynucleotides of the invention to reflect the codon usage of any particular host organism in which the polypeptides of the invention are to be expressed.

Any combination of the above mentioned degrees of homology and minimum sizes may be used to define polynucleotides of the invention, with the more stringent combinations (i.e. higher homology over longer lengths) being preferred. Thus for example a polynucleotide which is at least 80% homologous over 25, preferably 30 nucleotides forms one aspect of the invention, as does a polynucleotide which is at least 90% homologous over 40 nucleotides.

Polynucleotides of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as a DNA polynucleotide and primers according to the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. This will involve making a pair of primers (e.g. f about 15-30 nucleotides) to a region of the ATR gene which it is desired to clone, bringing the primers into contact with mRNA or cDNA obtained from a human cell (e.g. a dividing cell such as a peripheral blood leukocyte), performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Such techniques may be used to obtain all or part of the ATR sequence described herein. Genomic clones containing the ATR gene and its introns and promoter regions may also be obtained in an analogous manner, starting with genomic DNA from a human cell, e.g. a liver cell.

Although in general the techniques mentioned herein are well known in the art, reference may be made in particular to Sambrook et al. (Molecular Cloning: A Laboratory Manual, 1989).

Polynucleotides which are not 100% homologous to the sequences of the present invention but fall within the scope of the invention can be obtained in a number of ways.

Other human allelic variants of the ATR sequence described herein may be obtained for example by probing genomic DNA libraries made from a range of individuals, for example individuals from different populations.

In addition, other animal, particularly mammalian (e.g. mice, rats or rabbits), more particularly primate, homologues of ATR may be obtained and such homologues and fragments thereof in general will be capable of selectively hybridizing to Seq. ID No. 1. Such sequences may be obtained by probing cDNA libraries made from dividing cells or tissues or genomic DNA libraries from other animal species, and probing such libraries with probes comprising all or part of Seq. ID. 1 under conditions of medium to high stringency (for example 0.03M sodium chloride and 0.03M sodium citrate at from about 50° C. to about 60° C.).

Allelic variants and species homologues may also be obtained using degenerate PCR which will use primers designed to target sequences within the variants and homologues encoding conserved amino acid sequences. Conserved sequences can be predicted from aligning the ATR amino acid sequence with that of rad3. The primers will contain one or more degenerate positions and will be used at stringency conditions lower than those used for cloning sequences with single sequence primers against known sequences.

Alternatively, such polynucleotides may be obtained by site directed mutagenesis of the ATR sequences or allelic variants thereof. This may be useful where for example silent codon changes arc required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides. Further changes may be desirable to represent particular coding changes found in ATR which give rise to mutant ATR genes which have lost the checkpoint function. Probes based on such changes can be used as diagnostic probes to detect such ATR mutants.

The invention further provides double stranded polynucleotides comprising a polynucleotide of the invention and its complement.

Polynucleotides or primers of the invention may carry a revealing label. Suitable labels include radioisotopes such as ³²P or ³⁵S, enzyme labels, or other protein labels such as biotin. Such labels may be added to polynucleotides or primers of the invention and may be detected using by techniques known per se.

Polynucleotides or primers of the invention or fragments thereof labelled or unlabelled may be used by a person skilled in the art in nucleic acid-based tests for detecting or sequencing ATR in the human or animal body.

Such tests for detecting generally comprise bringing a human or animal body sample containing DNA or RNA into contact with a probe comprising a polynucleotide or primer of the invention under hybridizing conditions and detecting any duplex formed between the probe and nucleic acid in the sample. Such detection may be achieved using techniques such as PCR or by immobilizing the probe on a solid support, removing nucleic acid in the sample which is not hybridized to the probe, and then detecting nucleic acid which has hybridized to the probe. Alternatively, the sample nucleic acid may be immobilized on a solid sort, and the amount of probe bound to such a support can be detected. Suitable assay methods of this any other formats can be found in for example WO89/03991 and WO90/13667.

Test for sequence ATR include bringing a human or animal body sample containing target DNA or RNA into contact with a probe comprising a polynucleotide or primer of the invention under hybridizing conditions and determining the sequence by, for example the Sanger dideoxy chain termination method (see Sambrook et al.).

Such a method generally comprises elongating, in the presence of suitable reagents, the primer by synthesis of a strand complementary to the target DNA or RNA and selectively terminating the elongation reaction at one or more of an A, C, G or T/U residue; allowing strand elongation and termination reaction to occur; separating out according to size the elongated products to determine the sequence of the nucleotides at which selective termination has occurred. Suitable reagents include a DNA polymerase enzyme, the deoxynucleotides dATP, dCTP, dGTP and dTTP, a buffer and ATP. Dideoxynucleotides are used for selective termination.

Tests for detecting or sequencing ATR in the human or animal body may be used to determine ATR sequences within cells in individuals who have, or are suspected to have, an altered ATR gene sequence, for example within cancer cells including leukaemic cells and solid amours such as breast, ovary, lung, colon, pancreas, testes, liver, brain, muscle and bone tumours.

In addition, the discovery of ATR will allow the role of this gene in hereditary diseases to be investigated, in a manner analogous to the ATM gene. In general, this will involve establishing the status of ATR (e.g using PCR sequence analysis) in cells derived from patients with diseases that may be connected with damage to replicating cells, e.g. familial predisposition to cancer, chromosome breakage or instability phenotype or repair-damage sensitivity phenotype.

The probes of the invention may conveniently be packaged in the form of a test kit in a suitable container. In such kits the probe may be bound to a solid support where the assay format for which the kit is designed requires such binding. The kit may also contain suitable reagents for treating the sample to be probed, hybridizing the probe to nucleic acid in the sample, control reagents, instructions, and the like.

The present invention also provides polynucleotides encoding the polypeptides of the invention described below. Because such polynucleotides will be useful as sequences for recombinant production of polypeptides of the invention, it is not necessary for them to be selectively hybridizable to the sequence Seq. ID No. 1, although this will generally be desirable. Otherwise, such polynucleotides may be labelled, used, and made as described above if desired. Polypeptides of the invention are described below.

Particularly preferred polynucleotides of the invention are those derived from the lipid kinase domain of ATR, its allelic variants and species homologues. The lipid kinase domain is represented by nucleotides 7054 to 8011 of Seq. ID. 1. Polynucleotides of the invention which comprise this domain are particularly preferred. The term “lipids kinase domain” refers to a domain which has homology to other known lipid kinases, in particular the p110 subunit of PI-3 kinase, as determined by sequence alignments.

Other preferred polynucleotides of the invention those which comprise nucleotides encoding amino acids 181 to 302 of Seq. ID No. 2 (nucleotides 620 to 985 of Seq. ID No. 1), which is believed to be a leucine zipper region, a putative site of protein-protein interaction, and amino acids 1358 to 1366 (nucleotides 4151 to 4177), which is also conserved.

In an additional aspect, polynucleotides of the invention include those of Seq. ID No. 3 and fragments thereof capable of selectively hybridizing to this sequence other than the fragment consisting of nucleotides 2482 to 6599 in which the following changes have been made: Deletion of residues 2499, 2501, 2507 & 2509; insertion of C between 5918/5919.

Particularly preferred fragments include those comprising residues 6826 to 7334 (the lipid kinase domain) and the leucine zipper regions 1476 to 1625 and 2310 to 2357. Additionally, the fragment comprising the conserved region 3891 to 3917 is preferred. Such polypeptides and fragments may be made and used as described above.

B. Polypeptides.

Polypeptides of the invention include polypeptides in substantially isolated form which comprise the sequence set out in Seq ID No. 2.

Polypeptides further include variants of such sequences, including naturally occurring allelic variants and synthetic variants which are substantially homologous to said polypeptides. In this context, substantial homology is regarded as a sequence which has at least 70%, e.g. 80% or 90% amino acid homology (identity) over 30 amino acids with the sequence of Seq. ID No. 2 except for the lipid kinase domain and C-terminal portion (residues 2326 to 2644) where substantial homology is regarded as at least 80% homology, preferably 90% homology (identity) over 50 amino acids.

Polypeptides also include other those encoding ATR homologues from other species including animals such as mammals (e.g. mice, rats or rabbits), especially primates, and variants thereof as defined above.

Polypeptides of the invention also include fragments of the above mentioned full length polypeptides and variants thereof, including fragments of the sequence set out in Seq. ID No. 2.

Preferred fragments include those which include an epitope, especially an epitope. Suitable fragments will be at least about 5, e.g. 10, 12, 15 or 20 amino acids in size. Polypeptide fragments of the ATR protein and allelic and species variants thereof may contain one or more (e.g. 2, 3, 5, or 10) substitutions, deletions or insertions, including conserved substitutions.

Conserved substitutions may be made according to the following table indicates conservative substitutions, where amino acids on the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |
| OTHER | | N Q D E |

Variants of the polypeptides of the invention may also comprise polypeptides wherein one or more of the specified (i.e., naturally encoded) amino acids is deleted or replaced or wherein one or more nonspecified amino acids are added: (1) without loss of the kinase activity specific to the polypeptides of the invention; or (2) with disablement of the kinase activity specific to the polypeptides of the invention; or (3) with disablement of the ability to interact with members or regulators of the cell cycle checkpoint pathway.

Epitopes may be determined either by techniques such as peptide scanning techniques as described by Geysen et al. Mol. Immunol., 23; 709-715 (1986).

Polypeptides of the invention may be in a substantially isolated form. It will be understood that the polypeptide may be mixed with carriers or diluents which will not interfere with the intended purpose of the polypeptide and still be regarded as substantially isolated. A polypeptide of the invention may also be in a substantially purified form, in which case it will generally comprise the polypeptide in a preparation in which more than 90%, e.g. 95%, 98% or 99% of the polypeptide in the preparation is a polypeptide of the invention. Polypeptides of the invention may be modified for example by the addition of Histidine residues to assist their purification or by the addition of a signal sequence to promote their secretion from a cell.

A polypeptide of the invention may be labelled with a revealing label. The revealing label may be any suitable label which allows the polypeptide to be detected. Suitable labels include radioisotopes, e.g. ¹²⁵I, enzymes, antibodies, polynucleotides and linkers such as biotin. Labelled polypeptides of the invention may be used in diagnostic procedures such as immunoassays in order to determine the amount of a polypeptide of the invention in a sample. Polypeptides or labelled polypeptides of the invention may also be used in serological or cell mediated immune assays for the detection of immune reactivity to said polypeptides in animals and humans using standard protocols.

A polypeptide or labelled polypeptide of the invention or fragment thereof may also be fixed to a solid phase, for example the surface of an immunoassay well or dipstick.

Such labelled and/or immobilized polypeptides may be paged into kits in a suitable container along with suitable reagents, controls, instructions and the like.

Such polypeptides and kits may be used in methods of detection of antibodies to the ATR protein or its allelic or species variants by immunoassay.

Immunoassay methods are well known in the art and will generally comprise:
(a) providing a polypeptide comprising an epitope bindable by an antibody again said protein;
(b) incubating a biological sample with said polypeptide under conditions which allow for the formation of an antibody-antigen complex; and
(c) determining whether antibody-antigen complex comprising said polypeptide is formed.

Polypeptides of the invention may be may by synthetic means (e.g. as described by Geysen et al.) or recombinantly, as described below.

Particularly preferred polypeptides of the invention include those spanning or within the lipid kinase domain, namely from amino acids 2326 to 2644 of Seq. ID. 2, or sequences substantially homologous thereto. Fragments as defined above from this region are particularly preferred. The polypeptides and fragments thereof may contain amino acid alterations as defined above, including substitutions at one or more of positions 2475, 2480 and 2494, which correspond to the positions of the rad3 substitutions described in the examples below. Preferred substitutions include D2475A, N2480K and D2494E.

Polypeptides of the invention may be used in in vitro or in vivo cell culture systems to study the role of ATR as a checkpoint gene. For example, truncated or modified (e.g. modified in the lipid kinase domain) ATRs may be introduced into a cell to disrupt the normal checkpoint functions which occur in the cell.

The polypeptides of the invention may be introduced into the cell by in situ expression of the polypeptide from a recombinant expression vector (see below). The expression vector optionally carries an inducible promoter to control the expression of the polypeptide.

The use of mammalian host cells is expected to provide for such post-translational modifications (e.g., myristolation, glycosylation, truncation, lapidation and tyrosine, serine or threonine phosphorylation) as may be needed to confer optimal biological activity on recombinant expression products of the invention.

Such cell culture systems in which polypeptide of the invention are expressed may be used in assay systems to identify candidate substances which interfere or enhance checkpoint functions in the cell (see below).

In an additional aspect, polypeptides of the invention include the protein of Seq. ID No. 4 and fragments thereof from the region other than the fragment consisting of amino acids 713 to 1778. Particularly preferred fragments include those comprising residues 2082 to 2386 (the lipid kinase domain) and the leucine zipper regions 298 to 347 and 576 to 591. Additionally, the fragment comprising the conserved region 1103 to 1111 is preferred. Such polypeptides and fragments may be made and used as described above.

The invention also provides polypeptides substantially homologous to the protein of Seq. ID No. 4, and fragments thereof. In this context, substantial homology is regarded as a sequence which has at least 70%, e.g. 80% or 90% amino acid homology (identity) over 30 amino acids with the sequence of Seq. ID No. 4 except for the lipid kinase domain and C-terminal portion (residues 2082 to 2386) where substantial homology is regarded as at least 80%, preferably at least 90% homology (identity) over 50 amino acids.

C. Vectors.

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are described below in connection with expression vectors.

D. Expression Vectors.

Preferably, a polynucleotide of the invention in a vector is operably linked to a control sequence which is capable of providing for the expression of the coding sequence by the host cell, i.e. the vector is an expression vector.

The term “operably linked” refers to a juxtaposition wherein the components described are in a relationship permitting them to function in their intended manner. A control sequence “operably linked” to a coding sequence is ligated in such a way that expression of the coding sequence is achieved under condition compatible with the control sequences.

Such vectors may be transformed into a suitable host cell as described above to provide for expression of a polypeptide of the invention. Thus, in a further aspect the invention provides a process for preparing polypeptides according to the invention which comprises cultivating a host cell transformed or transfected with an expression vector as described above under conditions to provide for expression by the vector of a coding sequence encoding the polypeptides, and recovering the expressed polypeptides.

The vectors may be for example, plasmid, virus or phage vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used in vitro, for example for the production of RNA or used to transfect or transform a host cell. The vector may also be adapted to be used in vivo, for example in a method of gene therapy.

A further embodiment of the invention provides host cells transformed or transfected with the vectors for the replication and expression of polynucleotides of the invention. The cells will be chosen to be compatible with the said vector and may for example be bacterial, yeast, insect or mammalian.

Polynucleotides according to the invention may also be inserted into the vectors described above in an antisense orientation in order to provide for the production of antisense RNA. Antisense RNA or other antisense polynucleotides may also be produced by synthetic means. Such antisense polynucleotides may be used in a method of controlling the levels of ATR or its variants or species homologues.

Promoters and other expression regulation signals may be selected to be compatible with the host cell for which the expression vector is designed. For example, yeast promoters include S. cerevisiae GAL4 and ADH promoters, S. pombe nmt1 and adh promoter. Mammalian promoters include the metallothionein promoter which is can be included in response to heavy metals such as cadmium. Viral promoters such as the SV40 large T antigen promoter or adenovirus promoters may also be used. All these promoters are readily available in the art.

E. Antibodies.

The invention also provides monoclonal or polyclonal antibodies to polypeptides of the invention or fragments thereof. The invention further provides a process for the production of monoclonal or polyclonal antibodies to polypeptides of the invention. Monoclonal antibodies may be prepared by conventional hybridoma technology using the polypeptides of the invention or peptide fragments thereof, as immunogens. Polyclonal antibodies may also be prepared by conventional means which comprise inoculating a host animal, for example a rat or a rabbit, with a polypeptide of the invention or peptide fragment thereof and recovering immune serum.

In order that such antibodies may be made, the invention also provides polypeptides of the invention or fragments thereof haptenised to another polypeptide for use as immunogens in animals or humans.

Preferred antibodies of the invention will be capable of selectively binding the human ATR protein, that is with an affinity at least 10 fold, preferably at least 100 fold that of the rad3 protein. Such antibodies can be obtained by routine experimentation, e.g. selecting regions of ATR protein with sequences different from the corresponding regions of rad3, making peptides comprising such sequences and using such peptides as immunogens. Following production of antibodies the binding of said antibodies may be determined. Preferred antibodies of the invention include those capable of selectively binding the lipid kinase domain (as defined above) of the human ATR protein. In addition, antibodies which are capable of binding the human and yeast (S. pombe) lipid kinase domains with similar affinity, but not to the domains of the ATM family of proteins form a leer aspect of the invention. Such antibodies may be raised against peptides from the lipid kinase domains which correspond to regions found to be identical, or substantially identical, in the yeast and human genes.

For the purposes of this invention, the term “antibody”, unless specified to the contrary, includes fragments of whole antibodies which retain their binding activity for a tumour target antigen. Such fragments include Fv, F(ab′) and F(ab′)₂ fragments, as well as single chain antibodies. Furthermore, the antibodies and fragments thereof may be humanised antibodies, eg. as described in EP-A-239400.

Antibodies may be used in method of detecting polypeptides of the invention present in biological samples by a method which comprises:
(a) providing an antibody of the invention;
(b) incubating a biological sample with said antibody under conditions which allow for the formation of an antibody-antigen complex; and
(c) determining whether antibody-antigen complex comprising said antibody is formed.

Suitable samples include extracts from dividing cells, e.g leukocytes or cancer cells including leukaemic cells and solid tumours such as breast, ovary, lung, colon, pancreas, tests, liver, brain, muscle and bone tumours.

Antibodies of the invention may be bound to a solid support and/or packaged into kits in a suitable container along with suitable reagents, controls, instructions and the like.

F. Assays.

Abrogating cell cycle checkpoints is a potential strategy for developing or designing drugs for anti-cancer therapy, both as a novel treatment as such and as part of a combination therapy to enhance the specific toxicity of current chemotherapeutic agents. For example alkylating agents such as nitrogen mustards are used a chemotherapeutic agents which damage DNA in rapidly dividing cells, leading to cell death. The toxicity of such agents may be lessened by DNA repair and checkpoint mechanisms. Abrogating such mechanisms will thus enhance the effectiveness of therapeutic compounds designed to damage DNA. Abrogation of the ATR checkpoint will be especially useful where tumour cells have lost other checkpoint or damage response genes, since these other genes may be able to complement the loss of ATR function in non tumour cells, leading to an even greater enhancement in the effectiveness of the chemotherapeutic agent.

The lipid kinase activity of ATR is a target for developing anticancer compounds, since the results presented in the following examples indicate that the kinase domain is required for ATR function. Thus the present invention provides an assay method for screening candidate substances for and cancer therapy which comprises:
(a) providing a polypeptide of the invention which retains lipid kinase activity and a substrate for said kinase, under conditions and with reagents such that the kinase activity will act upon the substrate;
(b) bringing said polypeptide and substrate into contact with a candidate substance;
(c) measuring the degree of decrease in the kinase activity of the polypeptide, and
(d) selecting a candidate substance which provides a decrease in activity.

The assay may be carried out in vitro, for example in the wells of a microtitre dish. Such a format may be readily adapted for automation, allowing large numbers of candidate substances to be screened.

The substrate may be a protein or lipid substrate of natural or synthetic origin upon which the polypeptide of the invention will act. Usually, the polypeptide of the invention will phosphorylate the substrate.

Any suitable format for the assay may be used by those of skill in the art of throughput assays. Typically, the polypeptide of the invention which retains lipid kinase activity will be bound to a solid support in the presence of a substrate and cellular and other components which are usually required for activity. Labelled phosphate and a candidate substance will be added to the mixture simultaneously or sequentially in either order. After a suitable reaction time (usually a few minutes but in any event enough for phosphorylation of the substrate in the absence of candidate substance to occur) the amount of free phosphate is determined, e.g. by precipitation of phosphate. Candidate substances which inhibit kinase activity will inhibit the incorporation of free phosphate into the substrate and thus where free phosphate is found this is indicative of inhibition.

Other assay formats may be used by those skilled in the art.

The candidate substances may be used in an initial screen in batches of for example 10 compounds per reaction, and the compounds of those batches which show inhibition tested individually.

Suitable candidate substances include peptides, especially of from about 5 to 20 amino acids in size, based on the sequence of the kinase domain, or variants of such peptides in which one or more residues have been substituted as described above. Peptides from panels of peptides comprising random sequences or sequences which have been varied consistently to provide a maximally diverse panel of peptides may be used. Further candidate substances include kinase inhibitors which are small molecules such as cyclosporin-like and staurosporin-like compounds, or other compounds commercially available in panels of small molecule inhibitors.

Candidate substances which show activity in in vitro screens such as the above can then be tested in in vivo systems, such as yeast or mammalian cells which will be exposed to the inhibitor and tested for checkpoint activity.

We have also shown that Rad3 possesses protein kinase activity. Target substrates of Rad3 protein kinase activity may be identified by incorporating test compounds in assays for kinase activity. Rad3 protein is resuspended in kinase buffer and incubated either in the presence of absence of the test compound (e.g., casein, histone H1, or appropriate substrate peptide). Moles of phosphate transferred by the kinase to the test compound arc measured by autoradiography or scintillation counting. Transfer of phosphate to the test compound is indicative that the test compound is a substrate of the kinase.

Agents that modulate Rad3/ATR lipid kinase or Rad 3 protein kinase activity may be identified by incubating a test compound and Rad3/ATR immunopurified from cells naturally expressing Rad3/ATR, with Rad3/ATR obtained from recombinant procaryotic or eukaryotic cells expressing the enzyme, or with purified Rad3/ATR, and then determining the effect of the test compound on Rad3/ATR activity. The activity of the Rad3/ATR lipid kinase or Rad3 protein kinase domains can be measured by determining the moles of ³²P-phosphate transferred by the kinase from gamma-³²-P-ATP to either itself (autophosphorylation) or to an exogenous substrate such as a lipid or protein. The amount of phosphate incorporated into the substrate is measured by scintillation counting or autoradiography. An increase in the moles of phosphate transferred to the substrate in the presence of the test compound compared to the moles of phosphate transferred to the substrate in the absence of the test compound indicates that the test compound is an activator of said kinase activity. Conversely, a decrease in the moles of phosphate transferred to the substrate in presence of the test compound compared to the moles of phosphate transferred to the substrate in the absence of the test compound indicates that the modulator is an inhibitor of said kinase activity.

In a presently preferred assay, a Rad3/ATR antibody linked to agarose beads is incubated with a cell lysate prepared from host cells expressing Rad3/ATR. The beads are washed to remove proteins binding nonspecifically to the beads and the beads are then resuspended in a kinase buffer (such as 25 mM K-HEPES pH 7.7, 50 mM potassium chloride, 10 mM magnesium chloride, 0.1% Nonidet-P-40, 20% glycerol, 1 mM DTT). The reaction is initiated by the addition of 100 μM gamma-³²P-ATP (4 Ci/mM) and an exogenous substrate such as lipid or peptide, and the reaction is carried out at 30° C. for 10 minutes. The activity of the kinase is measured by determining the moles of ³²P-phosphate transferred either to the kinase itself or the added substrate. In a preferred embodiment the host cells lack endogenous Rad3/ATR kinase activity. The selectivity of a compound that modulates the lipid kinase activity of Rad3/ATR ran be evaluated by comparing its activity on Rad3/ATR to its activity on, for example, other known phosphatidylinositol-3 (PI-3) related-kinases. The combination of the recombinant Rad3/ATR products of the invention with other recombinant PI-3-related kinase products in a series of independent assays provides a system for developing selective modulators of Rad3/ATR kinase activity. Similarly, the selectivity of a compound that modulates the protein kinase activity of Rad3 may be determined with reference to other protein kinases, for example the DNA dependent protein kinase or ATM.

In addition, the demonstration that the rad mutant rad.D2249E (see Examples) can act as a dominant negative mutant indicates involvement in one or more protein complexes, and such complexes themselves can be targeted for therapeutic intervention. We have shown, for example, that Rad3 can both self associate and associate with ATR. It is therefore likely that Rad/ATR function as multimeric molecules. Mutant yeast rad or human ATR genes, or derivatives thereof which also lack rad/ATR activity may be introduced into cells to act as dominant negative mutants. Thus for example if expression of a dominant negative mutant (e.g. ATR D2475A, N2480K or D2494E) in a tumour cell leads to enhanced radiation sensitivity this indicates that the native ATR is still functioning and thus a target for therapeutic agents.

Interacting proteins including components of multimeric protein complexes involving Rad3 or ATR may be identified by the following assays.

A first assay contemplated by the invention is a two-hybrid screen. The two-hybrid system was developed in yeast (Chien et al. (1991)) and is based on functional in vivo reconstitution of a transcription factor which activates a reporter gene. Specifically, a polynucleotide encoding a protein that interacts with Rad3/ATR is isolated by: transforming or transfecting appropriate host cells with a DNA construct comprising a reporter gene under the control of a promoter regulated by a transcription factor having DNA a binding domain and an activating domain; expressing in the host cells a first hybrid DNA sequence encoding a first fusion of pan or all of Rad3/ATR and either the DNA binding domain or the activating domain of the transcription factor; expressing in the host cell a library of second hybrid DNA sequences encoding second fusion of part or all putative Rad3/ATR binding proteins and the DNA binding domain or activating domain of the transcription factor which is not incorporated in the first fusion; detecting binding of an Rad3/ATR interacting protein to Rad3/ATR in a particular host cell by detecting the production of reporter gene production the host cell; and isolating second hybrid DNA sequences encoding the interacting protein from the particular host cell. Presently preferred for use in the assay are a lexA promoter to drive expression of the reporter gene, the LacZ reporter gene, a transcription factor comprising the led DNA binding domain and the GAL4 transactivation domain, and yeast host cells.

Other assays for identifying proteins that interact with Rad3 or ATR may involve immobilising Rad3/ATR or a test protein, detectably labelling the nonimmobilised binding partner, incubating the binding partners together and determining the amount of label bound. Bound label indicates that the test protein interacts with Rad3/ATR.

Another type of assay for identifying Rad3 or ATR interacting proteins involves immobilising Rad3/ATR or a fragment thereof on a solid support coated (or impregnated with) a fluorescent agent, labelling a test protein with a compound capable of exciting the fluorescent agent, contacting the immobilised Rad3/ATR with the labelled test protein, detecting light emission by the fluorescent agent, and identifying interacting proteins as test proteins which result in the emission of light by the fluorescent agent. Alternatively, the putative interacting protein may be immobilised and Rad3/ATR may be labelled in the assay.

Compounds that modulate interaction between Rad3/ATR and other cellular components may be used in methods of treating cancer. For example, if a particular form of cancer results from a mutation in a gene other than ATR such as the p53 gene, an agent which inhibits the transcription or the enzymatic activity of ATR and thus the G₂ cell cycle checkpoint may be used to render cancerous cells more susceptible to chemotherapy or radiation therapy. The therapeutic value of such an agent lies in the fact that current radiation therapy or chemotherapy in most cases does nothing to overcome the ability of the p53 mutant cancerous cell to sense and correct the DNA damage imposed as a result of the treatment. As a result, a cancer cell can simply repair the DNA damage. Modulating agents of the invention may therefore be chemotherapy and radiation adjuvants or may be directly active as chemotherapy drugs themselves.

Assays for identifying compounds that modulate interaction of Rad3/ATR with other proteins may involve: transforming or transfecting appropriate host cells with a DNA construct comprising a reporter gene under the control of a promoter regulated by a transcription factor having a DNA-binding domain and an activating domain: expressing in the host cells a first hybrid DNA sequence encoding a first fusion of part or all of Rad3/ATR and the DNA binding domain or the activating domain of the transcription factor; expressing in the host cells a second hybrid DNA sequence encoding part or all of a protein that interact with Rad3/ATR and the DNA binding domain or activating domain of the transcription factor which is not incorporated in the first fusion; evaluating the effect of a test compound on the interaction between Rad3/ATR and the interacting protein by detecting binding of the interacting protein to Rad3/ATR in a particular host cell by measuring the production of reporter gene product in the host cell in the presence or absence of the test compound; and identifying modulating compounds as those test compounds altering production of the reported gene product in comparison to production of the reporter gene product in the absence of the modulating compound. Presently preferred for use in the assay are a lexA promoter to drive expression of the reporter gene, the lacZ reporter gene, a transcription factor comprising the lexA DNA domain and the GALA transactivation domain, and yeast host cells.

Another type of assay for identifying compounds that modulate the interaction between Rad3/ATR and an interacting protein involves immobilising Rad3/ATR or a natural Rad3/ATR interacting protein, delectably labelling the nonimmobilised binding partner, incubating the binding partners together and determining the effect of a test compound on the amount of label bound wherein a reduction in the label bound in the present of the test compound compared to the amount of label bound in the absence of the test compound indicates that the test agent is an inhibitor of Rad3/ATR interaction with the protein. Conversely, an increase in the bound in the presence of the test compared to the amount label bound in the absence of the compared indicates that the putative modulator is an activator of Rad3/ATR interaction with the protein.

Yet another method contemplated by the invention for identifying compounds that modulate the binding between Rad3/ATR and an interacting protein involves immobilising Rad3/ATR or a fragment thereof n a solid support coated (or impregnated with) a fluorescent agent, labelling the interacting protein with a compound capable of exciting the fluorescent agent, contacting the immobilised Rad3/ATR with the labelled interacting protein in the presence and absence of a test compound, detecting light emission by the fluorescent agent, and identifying modulating compounds as those test compounds that affect the emission of light by the fluorescent agent in comparison to the emission of light by the fluorescent agent in the absence of the test compound. Alternatively, the Rad3/ATR interacting protein may be immobilised and Rad3/ATR may be labelled in the assay.

We have shown that Rad3 interacts with ATR. Therefore the above-mentioned assays may also be used to identify compounds that modulate the interaction between Rad3 and ATR where the interacting protein described in the assay methods is either Rad3 or ATR.

We have also shown that Rad3 can bind to itself, strongly suggesting that ATR can also bind to itself. Therefore the above-mentioned assays may also be used to identify compounds that modulate Rad3-Rad3 interactions and ATR-ATR interactions.

Such compounds could be used therapeutically to disrupt ATR-ATR interactions and increase the sensitivity of tumour cells to chemotherapy and/or radiotherapy. Thus the invention provides an assay method for screening candidate substances for anticancer therapy which comprises:
(a) (i) incubating a polypeptide of the invention with another polypeptide of the invention, which may be the same as or different to the first polypeptide, under conditions which allow the first polypeptide to bind to the second polypeptide to form a complex;
   (ii) bringing the complex thus formed into contact with a candidate substance; or
(a) incubating a polypeptide of the invention with another polypeptide of the invention, which may be the same as or different to the first polypeptide, under conditions which allow the first polypeptide to bind to the second polypeptide to form a complex and in the presence of a candidate substance; and
(b) determining whether the candidate substance inhibits binding of the first polypeptide to the second polypeptide and
(c) selecting a candidate substance which inhibits binding of the first polypeptide to the second polypeptide.

Preferably the first and second polypeptide may be distinguished from each other. For example, the first polypeptide and the second polypeptide may both be ATR, or may both be Rad3, or one may be ATR and one may be Rad3 or derivatives of either ATR or Rad3 which retain binding activity. When both polypeptides are ATR or Rad3, preferably two distinguishable forms of ATR/Rad3 would be used in these assays. They may be distinguished by, for example, labelling either of the polypeptides. Examples of labels include radioactive labels, epitope tags or other polypeptide tags such as glutathione-S-transferase. For example, one form of Rad3 may have one form of epitope tag, and the or form would have a different epitope tag, allowing them to be distinguished immunologically such that binding of one to the other can be ascertained quantitively or qualitatively. In a preferred method, the first polypeptide may be immobilised, for example to agarose beads or a solid support, and the second polypeptide may be in free solution. Binding is then determined using methods described above and well-known to skilled persons.

Also comprehended by the present invention are antibody products (e.g., monoclonal and polyclonal antibodies, single chain antibodies, chimeric antibodies, CDR-grafted antibodies and the like) and other binding proteins (such as those identified in the assays above) which are specific for the Rad3 protein kinase domain or the Rad3/ATR lipid kinase domains. Binding proteins can be developed using isolated natural or recombinant enzymes. The binding proteins are useful, in turn, for purifying recombinant and naturally occurring enzymes and identifying cells producing such enzymes. Assays for the detection and quantification of proteins in cells and in fluids may involve a single antibody substance or multiple antibody substances in a “sandwich” assay format. The binding proteins are also manifestly useful in modulating (i.e., blocking, inhibiting, or stimulating) enzyme/substrate or enzyme/regulator interactions.

Modulators of Rad3/ATR may affect its kinase activity, its localisation in the cell, and/or its interaction with members of the cell cycle checkpoint pathway. Selective modulators may include, for example, polypeptides or peptides which specifically bind to Rad3/AIR or Rad3/ATR nucleic acid, and/or other non-peptide compounds (e.g., isolated or synthetic organic molecules) which specifically react with Rad3/ATR or Rad/ATR nucleic acid. Mutant forms of Rad3/ATR which affect the enzymatic activity or cellular localisation of wild-type Rad3/ATR are also contemplated by the invention.

Furthermore, combinatorial libraries, peptide and peptide mimetics, defined chemical entities, oligonucleotides, and natural product libraries may be screened for activity as modulators of Rad3/ATR kinase activity and Rad3/ATR interactions in assays such as those described above.

F. Therapeutic Uses

Modulators of Rad3/ATR activity, including inhibitors of their lipid kinase and protein kinase activities, may be used in anti cancer therapy. In particular, they may be used to increase the susceptibility of cancer cells to chemotherapy and/or radiotherapy by virtue of their ability to disrupt the cell cycle regulatory functions of Rad/ATR.

Thus the invention provides the use of compounds that modulate Rad3/ATR activity, identified by the screening assays described above, in a method of treatment of cancer. In one embodiment, said compounds are capable of inhibiting rad3/ATR lipid kinase and/or Rad3 protein kinase activity. In another embodiment, said compounds are capable of inhibiting interactions between ATR and itself and/or between ATR and other interacting proteins which may, for example, normally form part of a multimeric protein complex.

It is to be understood that the term ‘compound’ in this context also refers to the candidate substances selected in the above-described assays.

Typically the compounds are formulated for clinical administration by mixing them with a pharmaceutically acceptable carrier or diluent. For example they can be formulated for topical, parenteral, intravenous, intramuscular, subcutaneous, intraocular or transdermal administration. Preferably, the compound is used in an injectable form. Direct injection into the pateient's tumour is advantageous because it makes it possible to concentrate the therapeutic effect at the level of the affected tissues. It may therefore be mixed with any vehicle which is pharmaceutically acceptable for an injectable formulation, preferably for a direct injection at the site to be treated. The pharmaceutically carrier or diluent may be, for example, sterile or isotonic solutions.

The dose of compound used may be adjusted according to various parameters, especially according to the compound used, the age, weight and condition of the patient to be treated, the mode of administration used, pathology of the tumour and the required clinical regimen. As a guide, the amount of compound administered by injection is suitably from 0.01 mg/kg to 30 mg/kg, preferably from 0.1 mg/kg to 10 mg/kg.

The routes of administration and dosages described are intended only as a guide since a skilled practitioner will be able to determine readily the optimum route of administration and dosage for any particular patient and condition.

Compounds to be administered may include polypeptides or nucleic acids. The nucleic acids may encode polypeptides or they may encode antisense constructs that inhibit expression of a cellular gene. Nucleic acids may be administered by, for example, lipofection or by viral vectors. For example, the nucleic acid may form part of a viral vector such as an adenovirus. When viral vectors are used, in general the dose administered is between 10⁴ and 10¹⁴ pfu/ml, preferably 10⁶ to 10¹⁰ pfu/ml. The term pfu (“plaque forming unit”) corresponds to the infectivity of a virus solution and is determined by infecting an appropriate cell culture and measuring, generally after 48 hours, the number of plaques of infected cells. The techniques for determining the pfu titre of a viral solution are well documented in the literature.

Any cancer types may be treated by dose methods, for example leukaemias, and solid tumours such as breat, ovary, lung, colon, pancreas, testes, liver, brain, muscle and bone amour. Preferably, the tumour has normal ATR function.

### DESCRIPTION OF THE DRAWINGS

FIG. 1

The relationship between ATR, rad3, mei-41, MEC1, TEL1 and ATM

A. Overall structure of ATR, Rad3, Mei-41, Mec1p, Tel1p and ATM.

Legend: open square—Rad3 domain; hated boxes—kinase domain

B. Dendrogram based on sequence alignments generated by the Clustal method (PAM250) using DNAstar software, rad31/ESR1/mei-41/ATR are more closely related to each other than to ATM and TEL1. Sequences of rad3 and ATM are available in the EMBL database.

The following examples illustrate the invention.

### EXAMPLE 1

The rad3 gene of S. pombe is one of six genes absolutely required for the DNA structure checkpoints in S. pombe (Al-Khodairy and Carr. 1992; Al-Khodairy et al. 1994). A sequence representing part of the rad3 gene was reported by Seaton et al. (1992). In attempting to clarify the intron/exon structure of this gene we identified sequencing anomalies at both the 5′ and 3′ ends. We have sequenced the complete gene (see Experimental Procedures) and find that rad3 is capable of encoding a product of 2386 amino acids. The C-terminal region contains the consensus sequences typical of a sub-class of kinases known as lipid kinases, the founder member of which is the p110 catalytic subunit of P13 kinase (Hiles et al. 1992).

A truncated rad3 clone lacking the amino terminus and the kinase region has been reported to complement the rad3::pR3H1.0 gene disruption mutant of rad3 (Jimenez er al. 1992). This disruption mutant does not remove the potential kinase domain. To clarify the role of this domain, we have created a null mutant by gene replacement. This mutant has amino acids 1477-2271 of rad3, including the kinase consensus domain, replaced by ura4⁺. This strain rad3.d, has identical checkpoint defects and radiation/hydroxyurea sensitivities to the rad3.136 mutant (Nasim and Smith, 1975) and the original rad3::pR3H1.0 disruption mutant (Jimenez et al. 1992: Seaton et al. 1992) (data not shown). We have created three separate point mutants in the putative kinase domain of rad3 and used these in gene replacement experiments to construct strains with defined kinase null mutations. All three strains, rad3.D2230A, rad3.N2235K and rad3.D2249E have phenotypes identical to the rat3.d null mutant (data not shown), suggesting that the kinase activity is required for Rad3 function. In the light of our findings, one interpretation of the results of Seaton et. al. (1992) and Jimenez et al. (1992) is that the partial clone may show intragenic complementation between the plasmid borne truncated gene and a genomic partial deletion which retains kinase function. Such an interpretation would be consistent with Rad3 acting as a dimer or multimer.

When the kinase null allele rad3.D2249E was moderately over-expressed in wild type cells under control of a modified nmt1 promoter (Maundrell, 1990), it caused extreme radiation sensitivity, assayed by UV strip tests, and acted as a dominant negative mutant (data not shown). When the same kinase null construct was expressed at a higher level, it inhibited growth (data not shown). Examination of the cells indicates that division continued very slowly, and at a smaller cell size wild type cells and cells containing empty vector divide at approximately 15 microns, while rad3 and rad3.D2249E over-expressing cells divide at approximately 11.2 microns (data not shown). In S. pombe, this usually indicates an advancement of mitosis.

The Human Rad3 Homolog, ATR

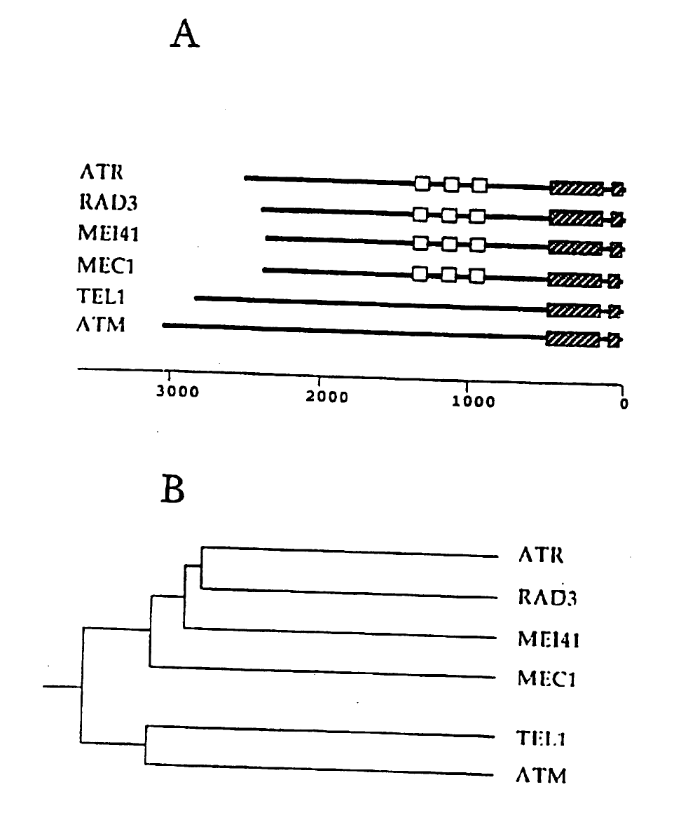

To identify a human form of rad3, a combination of methods was applied. Through these approaches, we have cloned the entire coding region of a human gene (see materials and methods), which we have named ATR (ataxia and rad related). ATR is capable of encoding a 2644 amino acid protein which is much more closely related to the products of S. pombe rad3, S. cerevisiae ESR (Kato and Ogawa, 1994) and D. melanogaster mei-41 genes (Hari et al. 1995) than to the human ATM and S. cerevisiae Tel1 proteins (Savitsky et al. 1995; Greenwell et al. 1995) and is likely to be the true homolog of rad3. ESR1 is allelic to the mec1/sad3 checkpoint mutants (Allen et al. 1994; Weinert et al. 1994) which have an equivalent phenotype to rad3. ATR is less closely related to the human checkpoint gene ATM, containing C-terminal putative lipid kinase domain and having a similar overall structure. Sequence alignments demonstrate clearly that the rad3/ESR1(MEC1/SAD3)/mei-41/ATR genes are more closely related to each other than any are to ATM or TEL1, and that ATM is more homologous to TEL1 (FIG. 1).

The ATM gene is expressed in a wide variety of tissues (Savitsky et. al. 1995). In S. cerevisiae, ESR1 shows low level expression in mitotic cells but is rapidly induced during meiosis I (Kato and Ogawa, 1994). Using Northern blot analysis, we have demonstrated that ATR is also weakly expressed in many tissues but that it is more highly expressed in testis (data not shown). Given that ATR, Rad3 and Esr1p proteins are more highly related to each other than to ATM, the higher ATR expression in testis is consistent with the observation that Esr1p has a role in meiotic recombination (Kato and Ogawa, 1994). Using FISH and PCR analysis, we have mapped ATR to chromosome 3q22-3q25 (data not shown). This region is not associated with known cancer prone syndromes.

In order to further investigate the possibility that Rad3 acts as a multimer, we have created two separate tagged constructs of full length rad3 in pREP based inducible vectors. In one, Rad3 is translated with two myc epitope tags at the N terminus, while in the other these are substituted for a triple HA epitope tag. When both constructs are expressed together in wild type cells, it is possible to co-precipitate the HA tagged Rad3 with the myc specific antibody, and the myc tagged Rad3 with the HA specific antibody (data not shown). This demonstrates that, in vivo, the Rad3 protein is capable of self association and is fully consistent with the complementation data of Jimenez et al. (1992).

Although the ATR gene could not complement the phenotype of the rad3 mutants, we have investigated the ability of ATR to form a protein complex with S. pombe Rad3 by expressing both ATR and myc-tagged S. pombe Rad3 in the same yeast cells. Using an anti-ATR antibody (which does not precipitate S. pombe Rad3, see materials and methods) we are able to co-precipitate the yeast protein. We were also able to precipitate the human ATR protein with myc-specific antibodies that recognise the S. pombe Rad3 (data not shown). These data suggest the human and yeast proteins can form a heteromeric-complex, which supports the contention, based on the sequence similarity, of a close functional relationship between these homologues.

Rad3 Proteins Have Associated Kinase Activity

Since mutagensis experiments suggest that the kinase activity of the Rad3 proteins in vivo appears to be essential for their function, we have investigated this activity further. Using S. pombe rad3::ura4 cells expressing HA tagged S. pombe Rad3, we have been able to detect a significant protein kinase activity which precipitates with HA-specific antibodies only when Rad3 is induced and which is not changed following irradiation (data not shown). This activity, which is specific to Rad3 or co-precipitating kind appears to reflect phosphorylation of Rad3 itself, since the major band above 200 kD that is phosphorylated can be detected by Western analysis with anti-HA antibody (data not shown). Attempts to identify convenient in vitro substrates such as myelin basic protein, RP-A and several purified S. pombe checkpoint proteins have so far proved unsuccessful. When the IP in vitro kinase assay is performed with cells over-expressing a ‘kinase-null’ D2249E version of Rad3, the associated kinase activity precipitated by HA-specific antibody is significantly reduced (data not shown). There are several possible explanations for this. The measured kinase activity could reflect Rad3 activity directly. In this case the residual activity seen with the kinase dead Rad3 could reflect the fact that it is not unknown for the equivalent D to E mutation in other protein kinases to produce a biologically inert protein with residual in vitro biochemical activity. Alternatively the kinase activity which phosphorylates Rad3 my be due to associated proteins, and these may interact less effectively with the D2249E mutant protein.

Discussion

The checkpoint pathways controlling cell cycle progression following DNA damage or interference in the individual events which comprise the cycle are of considerable importance in maintaining genetic stability and can be considered as pathways which suppress tumorgenesis. Several tumour suppressor genes are intimately involved in subsets of the checkpoint pathways (reviewed in Hartwell and Kastan, 1994), particularly those affecting the transition from G1 into S phase and commitment to the cell cycle. The convergence of the two yeast model systems for checkpoints clearly indicates that the genes involved in these pathways are conserved. Our work extends this conservation to metazoan cells, and clarifies the relationship between rad3. ESR1(MEC1/SAD3), mei-41 and the ATM gene.

In is work we demonstrate that the correct sequence of the rad3 gene places its product in the family of protein/lipid kinases related to ATM. Over-expression of kinase-defective rad3 mutant in S. pombe causes a dominant negative phenotype, which suggest that Rad3 is acting as a member of a protein complex whose integrity is necessary for checkpoint function. This is consistent with the observation that rad1, rad9, rad17, rad26 and hus1 deletion mutants all have phenotypes indistinguishable from ra3.d (Sheldrick and Carr, 1993). Unexpectedly, unlike the remaining checkpoint rad genes, high level over-expression of either wild type or mutant rad3 alleles inhibits cell growth and causes mitosis to occur at a reduced cell size, indicative of premature entry into mitosis. This ‘semi wee’ phenotype is not observed in the null mutant, and may indicate interference in a second pathway whose function overlaps with that of Rad3 and acts to inhibit mitosis. A candidate for such a pathway is the ATM/TEL1 pathway which has been shown to have some overlapping functions with the ESR1(MEC1/SAD1) pathway (Morrow et al. 1995).

The structure of ATM is most closely related to the Tel1p, which is involved in maintaining telomere length (Greenwell et al., 1995). However, ATM function also appears related to that of the Rad3/Esr1p/mei-41 products. Following the initial discovery of the ATM gene and its sequence relationship to the rad3/ESR1 genes and to TEL1, it was not clear whether, as in many cases in yeast, the gene had duplicated and diverged in yeast, or whether the two yeast proteins defined conserved sub-families of closely related genes. The significant finding of this work is the identification of a human gene, ATR, which is more closely related to rad3/ESR1/mei-41. This defines two structurally distinct checkpoint related subfamilies of protein/lipid kinases that are conserved throughout eukaryotic evolution. Although the proteins in these two subfamilies may have some overlapping functions, they probably control different processes. For example: the rad3 sub-family in yeast control all the G1 and G2 DNA damage checkpoints in response to both uv and ionising radiation, and the S phase checkpoint which prevents mitosis following inhibition of replication (Al-Khodairy and Carr, 1992; Allen et al., 1994; Weinert et al., 1994). In contrast. A-T cells have abnormal responses to a narrow range of DNA damaging agents including ionising radiation, bleomycin and neocarzinostatin, which produce strand breaks in DNA as a consequence of radical attack. The response to uv and most chemical carcinogens is normal, as is the response to the inhibition of DNA synthesis. It is possible that some or all of the remaining DNA damage checkpoints and the S phase checkpoint are controlled by ATR.

Experimental Procedures

Strains, Plasmids and Media

Standard genetic techniques, growth conditions and media for S. pombe are described in Gutz et al. (1974). S. pombe strain sp011 (ura4.D18, leu1.32 ade6.704 h) has been described previously (Murray et al. 1992). Plasmid pSUB41 was a gift from S. Subramani (Seaton et al. 1992).

Cloning of S. pombe rad3

A 4.0 kb Kpn1 fragment was excised from pSUB41 and sequenced in both directions to obtain the 5′ rad3 sequence. The 3′ clone was identified from a genomic library (Barbet et al. 1992) by colony hybridisation using a 1 kb 3′ probe derived from the published rad3 sequence, and sequenced in both directions. In this way, the sequence of die entire rad3 gene was assembled.

Null and “Kinase Dead” Rad3 Mutants

A construct of rad3, in which the 794 amino acids between aa 1477 and aa2271 (including the kinase domain) were replaced with a ura4+ gene, was created using the methodology described in Barbet et al. (1992). A linear fragment of this was used to transform sp011 to uracil prototropy and single copy integration at the rad3 locus was checked by Southern blotting. To create the site specific kinase null mutations, a C-terminal 3.01 kb BamHI-SalI fragment of rad3 was mutated with either (A: GTTTTCGCCATGGCGCGCTCCCAAACCCAA, B: TTCATCAAACAATATCTTTTCGCCATGGCG, or C: CAAAAAGAGACAGTTGAATTCGACATGGATAG) in order to introduce either the D2230A, N2235K or D2249E mutations into the kinase domain. Analogous changes have previously been used in the analysis of P13 kinase VPS34 of S. cerevisiae (Schu et al. 1993). These fragments were then used to transform the rad3.d null mutant and gene replacements selected by their ability to grow on FOA containing media (Grimm et al. 1988). All strains were checked by Southern blotting. Full length expression constructs of rad3.D2230A were created in pREP1 and pREP41 (Maundrell, 1990) by standard subcloning following introduction of an NdeI site at the ATG and deletion of three internal NdeI sites.

UV Radiation Sensitivity Strip Tests

Expression from REP1 (high) and REP41 (intermediate) was induced by the absence of thiamine for 18 hours prior to plating. Plates were irradiated with a gradient of uv doses down the plate from 0 to 300 Jm⁻² according to the settings on a Stratagene Stratalinker.

Cloning and Expression of ATR

To isolate an appropriate probe for identifying cDNAs corresponding to a human rad3 homologue, degenerate oligonucleotides were designed against the amino acids LGL-GDRH (5′ oligo; oDH18) and HVDF[D/N]C (3′ oligo; oDH-16) of Rad3/Esr1p. Inosine was incorporated at positions of four-fold degeneracy, and primers were tailed with BamHI (oDH18) and EcoRI (oDH16) to facilitate cloning. DNA sequence analysis of the ˜100 bp PCR product obtained from amplification of peripheral blood leukocyte cDNA demonstrated significant similarity to MEC1/rad3. This sequence was used to synthesize a non-degenerate primer (oDH-23; GACGCAGAATTCACCAGTCAAAGAAATCAAAGAG) for PCR with an additional degenerate primer (oDH17) designed against the amino acid sequence KFPPII/VIIL/FIYIO/EIWF of Rad3/Esr1p. The 174 bp product of this reaction was used directly to screen a macrophage cDNA library. Four positive clones were isolated (the largest approximately 3 kb).

In parallel, database searches with full length S. pombe rad3 derived from the EMBL database a human cDNA clone, HSAAADPDG, as a potential homologue of rad3, if a single frameshift was allowed for in the 233 bp sequence. This 233 bp sequence is contained within a 1.6 kb clone obtained from Dr. N. Affara, Human Molecular Genetics Research Group, Cambridge University, UK The entire clone (1.6 kb) was sequenced and lies within the cDNA clones identified by degenerate PCR and library screens. To identify the whole gene, RACE PCR experiments were performed on cDNA derived from placental and thymus mRNA using the instructions provided with a Clontech Marathon Kit. Gene specific primers were derived from the cDNA clones. From these experiments, a 8239 bp cDNA sequence was assembled with an internal ORF of 2644 amino acids, a 79 bp 5′ noncoding region, a 194 bp 3′ noncoding region and a poly A⁺ tail. Parts of the sequence were determined solely by PCR. To avoid errors, clones from a minimum of 3 independent PCR reactions were sequenced in both directions.

The 233 bp sequence corresponds to the sequence of nucleotides 6809 to 7042 (234 nt in total) of Seq. ID No. 1 except for a single base deletion at position 6942. This sequence encodes amino acids 2244 to 2320 of Seq. ID No. 2.

The sequence of the “1.6 kb” insert corresponds to nucleotides 5725 to 7104 (1353 nt) of Seq. ID No. 1, and encodes amino acids 1892 to 2340 of Seq. ID No. 2.

Northern blot hybridisation: a 1.3 kb PCR product was amplified in the presence of ³²P-dCTP using primers 279-3 (TGGATGATGACAGCTTGTGTC) and 279-6 (TGTAGTCGCTGCTCAATGTC). A nylon membrane containing 2 μg of size-fractionated polyA+ RNA from a variety of human tissue sources (Clontech Laboratories) was probed as recommended by the manufacturer except that the final wash was performed at 55° C. rather than 50° C. to minimize the possibility of cross-hybridisation to related sequences.

Mapping ATR.

We mapped the ATR gene to chromosome 3 by a combination of fluorescent in situ hybridisation and polymerase chain reaction (PCR) based assays. FISH analysis using a cDNA clone identified the ATR gene on chromosome 3, at approximately position q22-23. PCR analysis also identified ATR on chromosome 3. Two primers (oATR23: GACGCAGAATTCACCAGTCAAAGAATCAAAGAG and oATR26: TGGTTTCTGAGAACATTCCCTGA) which amplify a 257 bp fragment of the ATR gene were used on DNA derived from human/rodent somatic cell hybrids containing various human chromosome panels available from the NIGMS Human Genetic Mutant Cell Repository (Drwinga et al. 1993). PCR with the same primers was used to sub-localise ATR to a specific region on chromosome 3. The templates for these amplifications consisted of DNA samples from patients with truncations along chromosome 3 (Leach et al. 1994).

Immunoprecipitation (IP) and Kinase Assays with Rad3

The S. pombe rad3 and human ATR genes were cloned into pREP41 expression vector for complementation studies. To tag the proteins, versions of these vectors containing in-frame N terminal tag sequences, either a double myc or a triple HA tag, were used (Griffiths et al. 1995). Tagged proteins were expressed by growing in media without thiamine (Maundrell. 1990). Yeast cells lysed in lysis buffer (25 mM Tris.Cl pH 7.5. 60 mM B-glycerophosphate, 0.1 mM Na₃VO₄, 1% Triton X-100, 50 mM MaCl, 2 mM EDTA. 50 mM NaF, 1 mM phenylmethylsulfonyl fluoride [PMSF], 5 μg/ml leupeptin, 5 μg/ml aprotinin, 1 mM DTT) by the addition of glass beads followed by treatment in a dismembrinator for 2 minutes For IP's 300 μg total protein extract was incubated on ice with the appropriate antibody for 30 min and the immune complexes precipitated by mixing with Protein G beads for a further 30 min at 4° C. For kinase assays, the immune complexes were washed 4 timed with Lysis buffer, once with Kinase Buffer (25 mM Hepes pH7.7; 50 mM KCl; 10 mM MgCl₂; 0.1% NP-40; 2% glycerol; 1 mM DTT), and incubated in Kinase Buffer with 10 μM ATP [50 Ci/mmol]) for 15 minutes at 30° C. The reactions were stopped with 20 ul 2×SDS sample buffer prior to separation on 6% polyacrylamide gels. Rad3 IP's contained several phosphorylated products, including one which comigrated with Rad3 protein itself on Western analysis.

### REFERENCES

Al-Khodairy. F., and. Carr, A. M. (1992). DNA repair mutants defining G2 checkpoint pathways in Schizosaccharomyces pombe. EMBO J. 11, 1343-1350.
Al-Khodairy, F., Fotou, E., Sheldrick, K. S., Griffiths, D. J. F., Lehmann, A. R. and Carr, A. M. (1994). Identification and characterization of new elements involved in checkpoints and feedback controls in fission yeast. Mol. Biol. Cell 5, 147-160.
Allen, J. B., Zhou, Z., Siede, W., Friedberg, E. C. and Elledge, S. J. (1994) The SAD1/RAD53 protein kinase controls multiple checkpoints and DNA damage-induced transcription in yeast. Genes Dev. 8, 2416-2428.
Barbet, N. C., Muriel, W. J., and Carr, A. M. (1992) Versatile shuttle vectors and genomic libraries for use with Schizosaccharomyces pombe. Gene 114, 59-66.
Bearnish, H. and Lavin, M. F. (1994) Radiosensitivity in ataxia-telangiectasia: anomalies in radiation-induced cell cycle delay. Int. J. Radial. Biol. 65, 175-184.
Carr, A. M. and Hoekstra, M. F. (1995) The Cellular Responses to DNA Damage. Trends in Cell Biology 5, 32-40.
Chien et al., (1991) Proc. Natl. Acad Sci USA 88, 9578-9582
Deng, C., Zhang, P., Harper, J. W., Elledge, S. J. and Leder, P. J. (1995) Mice lacking p21^{CIP1/WAF1} undergo normal development, but are defective in G1 checkpoint control Cell, (in press).
Drwinga, H. L., Tojia, L. H., Kim, C. H., Greene, A. E., and Mulovor, R. A. (1993). NIGMS Human/Rodent Somatic Cell Hybrid Mapping Panels 1 and 2. Genomics 16:311-314.
El-Deiry, W. S., Tokino, T., Velculescu, V. E., Levy, D. B., Parson, R., Trent, J. M., Lin. D., Mercer, W. E., Kinzler, K. W. and Vogelstein, B. (1993) WAF1, a potential mediator of p53 tumour suppression. Cell 75, 817-825
Enoch, T., Carr, A. M. and Nurse, P. (1992). Fission yeast genes involved in coupling mitosis to completion of DNA-replication. Genes Dev. 6, 2035-2046.
Greenwell, P. W., Kronmal S. L., Porter, S. E., Gassenhuber, J., Obermaier, B. and Petes, T. D. (1995) TEL1, a gene involved in controlling telomere length in Saccharomyces cerevisiae, is homologous to the human ataxia telangiectasia (ATM) gene. Cell submitted.
Grimm, C., Kholi, J. Murray, J. M. and Maundrell, K. (1988) Genetic engineering of Schizosaccharomyces pombe: a system for gene disruption and replacement using the ura4 gene as a selectable marker. Mol. Gen. Genet. 215, 81-86.
Gutz, H., Heslot, H. Leupold, U. and Loprieno, N. (1974). In “Handbook of Genetics”, King R. C., Ed. Plenum Press, New York, Vol. 1, 395-446.
Hari, K. L., Santerre. A., Sekelsky, J. J., McKim, K. S., Boyd, J. B. and Hawley, R. S. (1995) The mei-41 gene of Drosophila melanogaster is functionally homologous to the human ataxia telangiec.
Harper, J. W., Adami, G., Wei, N., Keyomarsi, K. and Elledge, S. J. (1993) The 21 kD Cdk interacting protein Cip1 is a potent inhibitor of G1 cyclin dependent kinases. Cell 75, 805-816.
Hartwell, L. H., and Kastan, M. B. (1994). Cell cycle control and Cancer. Science 266, 1821-1828.
Hiles, I. D., Otsu M., Volinia, S., Fry, M. J., Gout, I., Dhand, R., Panayotou, G., Ruiz-Larrea, F., Thompson, A., Totty, N. F., Hsuan, J. J., Courtneidge, S. A, Parker. P. J. and Waterfield M. D. (1992) Phosphatidylinositol 3-kinase: structure and expression of the 110 kd catalytic subunit. Cell 70, 419-429.
Jimenez, G., Yucel, J., Rowley, R. and Subramani S. (1992) The rad3+gene of Schizosaccharomyces pombe is involved in multiple checkpoint functions and in DNA repair. Proc Natl. Acad. Sci. USA 87, 4952-4956
Kato, R. and Ogawa, H. (1994) An essential gene, ESR1, is required for mitotic cell growth, DNA repair and Meiotic recombination in Saccharomyces cerevisiae. Nucleic Acids Res. 22, 3104-3112.
Lamb, J. R., Petit-Frere, C., Broughton, B. C., Lehmann, A. R. and Green, M. H. L. (1989) Inhibition of DNA replication by ionizing radiation is mediated by a trans acting factor. Int. J. Radiat. Biol. 56, 125-130.
Leach, R. J., Chinn, R., Reus, B. E., Hayes, S., Schantz L., Dubois, B., Overhauser, 37 Ballabio, A., Drabkin, H. Lewis, B. T., Mendgen, G., and Naylor, S. L. (1994) Regional Localisation of 188 Sequence Tagged Sites on a Somatic Cell Hybrid Mapping Panel for Human Chromosome 3 Genomics 24, 549-556
Maundrell, K. (1990), nmt1 of fission yeast. A highly transcribed gene completely repressed by thiamine. J. Biol. Chem. 265, 10857-10864.
Morrow, D. M., Tagle, D. A., Shiloh, Y., Collins F. S. and Hieter, P. (1995) HAT1/TEL1, a Saccharomyces cerevisiae homologue of the human gene mutated in ataxia-telangiectasia, is functionally related to the yeast checkpoint gene MEC1/ESR1. Cell submitted.
Murray, J. M., Doe, C. Schenk, P. Carr, A. M. Lehmann, A. R. and Watts, F. Z. (1992) Cloning and characterization of the S. pombe rad15 gene, a homologue to the S. cerevisiae RAD3 and human ERCC2 genes Nucleic Acids Res. 20, 2673-2678.
Nasim, A. and Smith, B. P. (1975) Genetic control of radiation sensitivity in Schizosaccharomyces pombe. Genetics 79, 573-582.
Painter, R. B. and Young, B. R. (1980) Radiosensitivity in ataxia-telangiectasia: A new explanation. Proc. Natl. Acad. Sci. USA. 77, 7315-7317
Rowley, R., Subramani, S. and Young, P. G. (1992). Checkpoint controls in Schizosaccharomyces pombe, rad1, EMBO J. 11, 1335-1342.
Savitsky, K., Bar-Shira, A., Gilad, S., Rotman, G., Ziv, Y., Vanagaite L., Tagle, D. A., Smith, S., Uziel, T., Sfez, S., Ashkenazi, M., Pecker, I., Frydman, M., Harnik, R., Patanjali, S. R., Simmons, A., Clines, G. A., Sartiel, A., Gatti, R. A., Chessa, L., Sanal, O., Lavine, M. F., Jaspers, N. G. J., Taylor, M. R., Arlett, C. F., Miki, T., Weissman, S. M., Lovett, M., Collins, F. S. and Shiloh, Y. (1995). A single ataxia telangiectasia gene with a product similar to PI-3 kinase. Science 286, 1749-1753.
Seaton, B. L., Yucel, J., Sunnerhagen P. and Subramani, S. (1992). Isolation and characterisation of the Schizosaccharomyces pombe rad3 gene which is involved in the DNA damage and DNA synthesis checkpoints. Gene 119, 83-89.
Schu, P. V., Takegawa, K., Fry, M. J., Stack, J. H., Waterfield, M. D. and Emr, S. D. (1993) Phosphatidylinositol 3-kinase encoded by yeast VPS34 gene essential for protein sorting. Science 260, 88-91.
Sheldrick, K. S. and Carr, A. M. (1993). Feedback controls and G2 checkpoints, fission yeast as a model system. BioEssays 15, 775-782.
Walworth, N., Davey, S. and Beach, D. (1993). Fission yeast chk1 protein kinase links the rad checkpoint pathway to cdc2. Nature 363, 368-371.
Weinert, T. A., and Hartwell, L. H. (1988). The RAD9 gene controls the cell cycle response to DNA damage in Saccharomyces cerevisiae. Science 241, 317-322.
Weinert, T. A., Kiser, G. L. Hartwell, L. H. (1994). Mitotic checkpoint genes in budding yeast and the dependence of mitosis on DNA replication and repair. Genes Dev. 8, 652-665.

Sequence Information.

| Sequence ID No. 1: ATR seq | | |
|---|---|---|
| 1 | GCGCTCTTCCGGCAGCGGTACGTTTGGAGACGCCGGGAACCCGCGTTGGCGTGGTTGACTAGTGCCTCGCAGCCT | 75 |
| 76 | CAGCATGGGGGAACATGGCCTGGAGCTGGCTTCCATGATCCCCGCCCTGCGGGAGCTGGGCAGTGCCACACCAGA | 150 |
| 151 | GGAATATAATACAGTTGTACAGAAGCCAAGACAAATTCTGTGTCAATTCATTGACCGGATACTTACAGATGTAAA | 225 |
| 226 | TGTTGTTGCTGTAGAACTTGTAAAGAAAACTGACTCTCAGCCAACCTCCGTGATGTTGCTTGATTTCATCCAGCA | 300 |
| 301 | TATCATGAAATCCTCCCCACTTATGTTTGTAAATGTGAGTGGAAGCCATGAGCGCAAAGGCAGTTGTATTGAATT | 375 |
| 376 | CAGTAATTGGATCATAACGAGACTTCTGCGGATTGCAGCAACTCCCTCCTGTCATTTGTTACACAAGAAAATCTG | 450 |
| 451 | TGAAGTCATCTGTTCATTATTATTTCTTTTTAAAAGCAAGAGTCCTGCTATTTTTGGGGTACTCACAAAAGAATT | 525 |
| 526 | ATTACAACTTTTTGAAGACTTGGTTTACCTCCATAGAAGAAATGTGATGGGTCATGCTGTGGAATGGCCAGTGGT | 600 |
| 601 | CATGAGCCGATTTTTAAGTCAATTAGATGAACACATGGGATATTTACAATCAGCTCCTTTGCAGTTGATGAGTAT | 675 |
| 676 | GCAAAATTTAGAATTTATTGAAGTCACTTTATTAATGGTTCTTACTCGTATTATTGCAATTGTGTTTTTTAGAAG | 750 |
| 751 | GCAAGAACTCTTACTTTGGCAGATAGGTTGTGTTCTGCTAGAGTATGGTAGTCCAAAAATTAAATCCCTAGCAAT | 825 |
| 826 | TAGCTTTTTAACAGAACTTTTTCAGCTTGGAGGACTACCAGCACAACCAGCTAGCACTTTTTTCAGCTCATTTTT | 900 |
| 901 | GGAATTATTAAAACACCTTGTAGAAATGGATACTGACCAATTGAAACTCTATGAAGAGCCATTATCAAAGCTGAT | 975 |
| 976 | AAAGACACTATTTCCCTTTGAAGCAGAAGCTTATAGAAATATTGAACCTGTCTATTTAAATATGCTGCTGGAAAA | 1050 |
| 1051 | ACTCTGTGTCATGTTTGAAGACGGTGTGCTCATGCGGCTTAAGTCTGATTTGCTAAAAGCAGCTTTGTGCCATTT | 1125 |
| 1126 | ACTGCAGTATTTCCTTAAATTTGTGCCAGCTGGGTATGAATCTGCTTTACAAGTCAGGAAGGTCTATGTGAGAAA | 1200 |
| 1201 | TATTTGTAAAGCTCTTTTGGATGTGCTTGGAATTGAGGTAGATGCAGAGTACTTGTTGGGCCCACTTTATGCAGC | 1275 |
| 1276 | TTTGAAAATGGAAAGTATGGAAATCATTGAGGAGATTCAATGCCAAACTCAACAGGAAAACCTCAGCAGTAATAG | 1350 |
| 1351 | TGATGGAATATCACCCAAAAGGCGTCGTCTCAGCTCGTCTCTAAACCCTTCTAAAAGAGCACCAAAACAGACTGA | 1425 |
| 1426 | GGAAATTAAACATGTGGACATGAACCAAAAGAGCATATTATGGAGTGCACTGAAACAGAAAGCTGAATCCCTTCA | 1500 |
| 1501 | GATTTCCCTTGAATACAGTGGCCTAAAGAATCCTGTTATTGAGATGTTAGAAGGAATTGCTGTTGTCTTACAACT | 1575 |
| 1576 | GACTGCTCTGTGTACTGTTCATTGTTCTCATCAAAACATGAACTGCCGTACTTTCAAGGACTGTCAACATAAATC | 1650 |
| 1651 | CAAGAAGAAACCTTCTGTAGTGATAACTTGGATGTCATTGGATTTTTACACAAAAGTGCTTAAGAGCTGTAGAAG | 1725 |
| 1726 | TTTGTTAGAATCTGTTCAGAAACTGGACCTGGAGGCAACCATTGATAAGGTGGTGAAAATTTATGATGCTTTGAT | 1800 |
| 1601 | TTATATGCAAGTAAACAGTTCATTTGAAGATCATATCCTGGAAGATTTATGTGGTATGCTCTCACTTCCATGGAT | 1875 |
| 1876 | TTATTCCCATTCTGATGATGGCTGTTTAAAGTTGACCACATTTGCCGCTAATCTTCTAACATTAAGCTGTAGGAT | 1950 |
| 1951 | TTCAGATAGCTATTCACCACAGGCACAATCACGATGTGTGTTTCTTCTGACTCTGTTTCCAAGAAGAATATTCCT | 2025 |
| 2026 | TGAGTGGAGAACAGCAGTTTACAACTGGGCCCTGCAGAGCTCCCATGAAGTAATCCGGGCTAGTTGTGTTAGTGG | 2100 |
| 2101 | ATTTTTTATCTTATTGCAGCAGCAGAATTCTTGTAACAGAGTTCCCAAGATTCTTATAGATAAAGTCAAAGATGA | 2175 |
| 2176 | TTCTGACATTGTCAAGAAAGAATTTGCTTCTATACTTGGTCAACTTGTCTGTACTCTTCACGGCATGTTTTATCT | 2250 |
| 2251 | GACAAGTTCTTTAACAGAACCTTTCTCTGAACACGGACATGTGGACCTCTTCTGTAGGAACTTGAAAGCCACTTC | 2325 |
| 2326 | TCAACATGAATGTTCATCTTCTCAACTAAAAGCTTCTGTCTGCAAGCCATTCCTTTTCCTACTGAAAAAAAAAAT | 2400 |
| 2401 | ACCTAGTCCAGTAAAACTTGCTTTCATAGATAATCTACATCATCTTTGTAAGCATCTTGATTTTAGAGAAGATGA | 2475 |
| 2476 | AACAGATGTAAAAGCAGTTCTTGGAACTTTATTAAATTTAATGGAAGATCCAGACAAAGATGTTAGAGTGGCTTT | 2550 |
| 2551 | TAGTGGAAATATCAAGCACATATTGGAATCCTTGGACTCTGAAGATGGATTTATAAAGGAGCTTTTTGTCTTAAG | 2625 |
| 2626 | AATGAAGGAAGCATATACACATGCCCAAATATCAAGAAATAATGAGCTGAAGGATACCTTGATTCTTACAACAGG | 2700 |
| 2701 | GGATATTGGAAGGGCCGCAAAAGGAGATTTGGTACCATTTGCACTCTTACACTTATTGCATTGTTTGTTATCCAA | 2775 |
| 2776 | GTCAGCATCTGTCTCTGGAGCAGCATACACAGAAATTAGAGCTCTGGTTGCAGCTAAAAGTGTTAAACTGCAAAG | 2850 |
| 2851 | TTTTTTCAGCCAGTATAAGAAACCCATCTGTCAGTTTTTGGTAGAATCCCTTCACTCTAGTCAGATGACAGCACT | 2925 |
| 2926 | TCCGAATACTCCATGCCAGAATGCTGACGTGCGAAAACAAGATGTGGCTCACCAGAGAGAAATGGCTTTAAATAC | 3000 |
| 3001 | GTTGTCTGAAATTGCCAACGTTTTCGACTTTCCTGATCTTAATCGTTTTCTTACTAGGACATTACAAGTTCTACT | 3075 |
| 3076 | ACCTGATCTTGCTGCCAAAGCAAGCCCTGCAGCTTCTGCTCTCATTCGAACTTTAGGAAAACAATTAAATGTCAA | 3150 |
| 3151 | TCGTAGAGAGATTTTAATAAACAACTTCAAATATATTTTTTCTCATTTGGTCTGTTCTTGTTCCAAAGATGAATT | 3225 |
| 3226 | AGAACGTGCCCTTCATTATCTGAAGAATGAAACAGAAATTGAACTGGGGAGCCTGTTGAGACAAGATTTCCAAGG | 3300 |
| 3301 | ATTGCATAATGAATTATTGCTGCGTATTGGAGAACACTATCAACAGGTTTTTAATGGTTTGTCAATACTTGCCTC | 3375 |
| 3376 | ATTTGCATCCAGTGATGATCCATATCAGGGCCCGAGAGATATCATATCACCTGAACTGATGGCTGATTATTTACA | 3450 |
| 3451 | ACCCAAATTGTTGGGCATTTTGGCTTTTTTTAACATGCAGTTACTGAGCTCTAGTGTTGGCATTGAAGATAAGAA | 3525 |
| 3526 | AATGGCCTTGAACAGTTTGATGTCTTTGATGAAGTTAATGGGACCCAAACATGTCAGTTCTGTGAGGGTGAAGAT | 3600 |
| 3601 | GATGACCACACTGAGAACTGGCCTTCGATTCAAGGATGATTTTCCTGAATTGTGTTGCAGAGCTTGGGACTGCTT | 3675 |
| 3676 | TGTTCGCTGCCTGGATCATGCTTGTCTGGGCTCCCTTCTCAGTCATGTAATAGTAGCTTTGTTACCTCTTATACA | 3750 |
| 3751 | CATCCAGCCTAAAGAAACTGCAGCTATCTTCCACTACCTCATAATTGAAAACAGGGATGCTGTGCAAGATTTTCT | 3825 |
| 3826 | TCATGAAATATATTTTTTACCTGATCATCCAGAATTAAAAAAGATAAAAGCCGTTCTCCAGGAATACAGAAAGGA | 3900 |
| 3901 | GACCTCTGAGAGCACTGATCTTCAGACAACTCTTCAGCTCTCTATGAAGGCCATTCAACATGAAAATGTCGATGT | 3975 |
| 3916 | TCGTATTCATGCTCTTACAAGCTTGAAGGAAACCTTGTATAAAAATCAGGAAAAACTGATAAAGTATGCAACAGA | 4050 |
| 4051 | CAGTGAAACAGTAGAACCTATTATCTCACAGTTGGTGACAGTGCTTTTGAAAGGTTGCCAAGATGCAAACTCTCA | 4125 |
| 4126 | AGCTCGGTTGCTCTGTGGGGAATGTTTAGGGGAATTGGGGGCGATAGATCCAGGTCGATTAGATTTCTCAACAAC | 4200 |
| 4201 | TGAAACTCAAGGAAAAGATTTTACATTTGTGACTGGAGTAGAAGATTCAAGCTTTGCCTATGGATTATTGATGGA | 4275 |
| 4276 | GCTAACAAGAGCTTACCTTGCGTATGCTGATAATAGCCGAGCTCAAGATTCAGCTGCCTATGCCAGCCAGGAGTT | 4350 |
| 4351 | GCTTTCTATTTATGACTGTAGAGAGATGGAGACCAACGGCCCAGGTCACCAATTGTGGAGGAGATTTCCTGAGCA | 4425 |
| 4426 | TGTTCGGGAAATACTAGAACCTCATCTAAATACCAGATACAAGAGTTCTCAGAAGTCAACCGATTGGTCTGGAGT | 4500 |
| 4501 | AAAGAAGCCAATTTACTTAAGTAAATTGGGTAGTAACTTTGCAGAATGGTCAGCATCTTGGGCAGGTTATCTTAT | 4575 |
| 4576 | TACAAAGGTTCGACATGATCTTGCCAGTAAAATTTTCACCTGCTGTAGCATTATGATGAAGCATGATTTCAAAGT | 4650 |
| 4651 | GACCATCTATCTTCTTCCACATATTCTGGTGTATGTCTTACTGGGTTGTAATCAAGAAGATCAGCAGGAGGTTTA | 4725 |
| 4726 | TGCAGAAATTATGGCAGTTCTAAAGCATGACGATCAGCATACCATAAATACCCAAGACATTGCATCTGATCTGTG | 4800 |
| 4801 | TCAACTCAGTACACAGACTGTGTTCTCCATGCTTGACCATCTCACACAGTGGGCAAGGCACAAATTTCAGGCACT | 4875 |
| 4876 | GAAAGCTGAGAAATGTCCACACAGCAAATCAAACAGAAATAAGGTAGACTCAATGGTATCTACTGTGGATTATGA | 4950 |
| 4951 | AGACTATCAGAGTGTAACCCGTTTTCTAGACCTCATACCCCAGGATACTCTGGCAGTAGCTTCCTTTCGCTCCAA | 5025 |
| 5026 | AGCATACACACGAGCTGTAATGCACTTTGAATCATTTATTACAGAAAAGAAGCAAAATATTCAGGAACATCTTGG | 5100 |
| 5101 | ATTTTTACAGAAATTGTATGCTGCTATGCATGAACCTGATGGAGTGGCCGGAGTCAGTGCAATTAGAAAGGCAGA | 5175 |
| 5176 | ACCATCTCTAAAAGAACAGATCCTTGAACATGAAAGCCTTGGCTTGCTGAGGGATGCCACTGCTTGTTATGACAG | 5250 |
| 5251 | GGCTATTCAGCTAGAACCAGACCAGATCATTCATTATCATGGTGTAGTAAAGTCCATGTTAGGTCTTGGTCAGCT | 5325 |
| 5326 | GTCTACTGTTATCACTCAGGTGAATGGAGTGCATGCTAACAGGTCCGAGTGGACAGATGAATTAAACACGTACAG | 5400 |
| 5401 | AGTGGAAGCAGCTTGGAAATTGTCACAGTGGGATTTGGTGGAAAACTATTTGGCAGCAGATGGAAAATCTACAAC | 5475 |
| 5476 | ATGGAGTGTCAGACTGGGACAGCTATTATTATCAGCCAAAAAAAGAGATATCACAGCTTTTTATGACTCACTGAA | 5550 |
| 5551 | ACTAGTGAGAGCAGAACAAATTGTACCTCTTTCAGCTGCAAGCTTTGAAAGAGGCTCCTACCAACGAGGATATGA | 5625 |
| 5626 | ATATATTGTGAGATTGCACATGTTATGTGAGTTGGAGCATAGCATCAAACCACTTTTCCAGCATTCTCCAGGTGA | 5700 |
| 5701 | CAGTTCTCAAGAAGATTCTCTAAACTGGGTAGCTCGACTAGAAATGACCCAGAATTCCTACAGAGCCAAGGAGCC | 5775 |
| 5776 | TATCCTGGCTCTCCGGAGGGCTTTACTAAGCCTCAACAAAAGACCAGATTACAATGAAATGGTTGGAGAATGCTG | 5850 |
| 5851 | GCTGCAGAGTGCCAGGGTAGCTAGAAAGGCTGGTCACCACCAGACAGCCTACAATGCTCTCCTTAATGCAGGGGA | 5925 |
| 5926 | ATCACGACTCGCTGAACTGTACGTGGAAAGGGCAAAGTGGCTCTGGTCCAAGGGTGATGTTCACCAGGCACTAAT | 6000 |
| 6001 | TGTTCTTCAAAAAGGTGTTGAATTATGTTTTCCTGAAAATGAAACCCCACCTGAGGGTAAGAACATGTTAATCCA | 6075 |
| 6076 | TGGTCGAGCTATGCTACTAGTGGGCCGATTTATGGAAGAAACAGCTAACTTTGAAAGCAATGCAATTATGAAAAA | 6150 |
| 6151 | ATATAAGGATGTGACCGCGTGCCTGCCAGAATGGGAGGATGGGCATTTTTACCTTGCCAAGTACTATGACAAATT | 6225 |
| 6226 | GATGCCCATGGTCACAGACAACAAAATGGAAAAGCAAGGTGATCTCATCCGGTATATAGTTCTTCATTTTGGCAG | 6300 |
| 6301 | ATCTCTACAATATGGAAATCAGTTCATATATCAGTCAATGCCACGAATGTTAACTCTATGGCTTGATTATGGTAC | 6375 |
| 6376 | AAAGGCATATGAATGGGAAAAAGCTGGCCGCTCCGATCGTGTACAAATGAGGAATGATTTGGGTAAAATAAACAA | 6450 |
| 6451 | GGTTATCACAGAGCATACAAAGTATTTAGCTCCATATCAATTTTTGACTGCTTTTTCACAATTGATCTCTCGAAT | 6525 |
| 6525 | TTGTCATTCTCACGATGAAGTTTTTGTTGTCTTGATGGAAATAATAGCCAAAGTATTTCTAGCCTATCCTCAACA | 6600 |
| 6601 | AGCAATGTGGATGATGACAGCTGTGTCAAAGTCATCTTATCCCATGCGTGTGAACAGATGCAAGGAAATCCTCAA | 6675 |
| 6676 | TAAAGCTATTCATATGAAAAAATCCTTAGAGAAGTTTGTTGGAGATGCAACTCGCCTAACAGATAAGCTTCTAGA | 6750 |
| 6751 | ATTGTGCAATAAACCGGTTGATGGAAGTAGTTCCACATTAAGCATGAGCACTCATTTTAAAATGCTTAAAAAGCT | 6825 |
| 6826 | GGTAGAAGAAGCAACATTTAGTGAAATCCTCATTCCTCTACAATCAGTCATGATACCTACACTTCCATCAATTCT | 6900 |
| 6901 | GGGTACCCATGCTAACCATGCTAGCCATGAACCATTTCCTGGACATTGGGCCTATATTGCAGGGTTTGATGATAT | 6975 |
| 6976 | GGTGGAAATTCTTGCTTCTCTTCAGAAACCAAAGAAGATTTCTTTAAAAGGCTCAGATGGAAAGTTCTACATCAT | 7050 |
| 7051 | GATGTGTAAGCCAAAAGATGACCTGAGAAAGGATTGTAGACTAATGGAATTCAATTCCTTGATTAATAAGTGCTT | 7125 |
| 7126 | AAGAAAAGATGCAGAGTCTCGTAGAAGAGAACTTCATATTCGAACATATGCAGTTATTCCACTAAATGATGAATG | 7200 |
| 7201 | TGGGATTATTGAATGGGTGAACAACACTGCTGGTTTGAGACCTATTCTGACCAAACTATATAAAGAAAAGGGAGT | 7275 |
| 7276 | GTATATGACAGGAAAAGAACTTCGCCAGTGTATGCTACCAAAGTCAGCAGCTTTATCTGAAAAACTCAAAGTATT | 7350 |
| 7351 | CCGAGAATTTCTCCTGCCCAGGCATCCTCCTATTTTTCATGAGTGGTTTCTGAGAACATTCCCTGATCCTACATC | 7425 |
| 7426 | ATGGTACAGTAGTAGATCAGCTTACTGCCGTTCCACTGCAGTAATGTCAATGGTTGGTTATATTCTGGGGCTTGG | 7500 |
| 7501 | AGACCGTCATGGTGAAAATATTCTCTTTGATTCTTTGACTGGTGAATGCGTACATGTAGATTTCAATTGTCTTTT | 7575 |
| 7576 | CAATAAGGGAGAAACCTTTGAAGTTCCAGAAATTGTGCCATTTCGCCTGACTCATAATATGGTTAATGGAATGGG | 7650 |
| 7651 | TCCTATGGGAACAGAGGGTCTTTTTCGAAGAGCATGTGAAGTTACAATGAGGCTGATGCGTGATCAGCGAGAGCC | 7725 |
| 7726 | TTTAATGAGTGTCTTAAAGACTTTTCTACATGATCCTCTTGTGGAATGGAGTAAACCAGTGAAAGGGCATTCCAA | 7800 |
| 7801 | AGCGCCACTGAATGAAACTGGAGAAGTTGTCAATGAAAAGGCCAAGACCCATGTTCTTGACATTGAGCAGCGACT | 7875 |
| 7876 | ACAAGGTGTAATCAAGACTCGAAATAGAGTGACAGGACTGCCGTTATCTATTGAAGGACATGTGCATTACCTTAT | 7950 |
| 7951 | ACAAGAAGCTACTGATGAAAACTTACTATGCCAGATGTATCTTGGTTGGACTCCATATATGTGAAATGAAATTAT | 8025 |
| 9026 | GTAAAAGAATATGTTAATAATCTAAAAGTAATGCATTTGGTATGAATCTGTGGTTGTATCTGTTCAATTCTAAAG | 8100 |
| 8101 | TACAACATAAATTTACGTTCTCAGCAACTGTTATTTCTCTCTGATCATTAATTATATGTAAAATAATATACATTC | 8175 |
| 8176 | AGTTATTAAGAAATAAACTGCTTTCTTAATAAAAAAAAAAAAAAAAAAAAAAAAAAAAAAAAAA | 8239 |
| Sequence ID No. 2: ATR protein | | |
| 1 | MGEHGLELASMIPALRELGSATPEEYNTVVQKPRQILCQFIDRILTDVNVVAVELVKKTDSQPTSV | 66 |
| 67 | MLLDFIQHIMKSSPLMFVNVSGSHERKGSCIEFSNWIITRLLRIAATPSCHLLHKKICEVICSLLFLFKSKSPAI | 141 |
| 142 | FGVLTKELLQLFEDLVYLHRRNVHGHAVEWPVVMSRFLSQLDIHMGYLQSAPLQLMSMQNLEFIEVTLLMVLTRI | 216 |
| 217 | IAIVFFRRQELLLWQIGCVLLEYGSPKIKSLAISFLTELFQLGGLPAQPASTFFSSFLELLKHLVEMDTDQLKLY | 291 |
| 292 | EEPLSKLIKTLFPFEAEAYRNIEPVYLNNLLEKLCVNFEDGVLMRLKSDLLKAALCHLLQYFLKFVPAGYESALQ | 366 |
| 367 | VRKVYVRNICKALLDVLGIEVDAEYLLGPLYAALKMESMEIIEEIQCQTQQENLSSNSDGISPKRRRLSSSLNPS | 441 |
| 442 | KRAPKQTEEIKHVDMNQKSILWSALKQKAESLQISLEYSGLKNPVIEMLEGIAVVLQLTALCTVHCSHQNMNCRT | 516 |
| 517 | FKDCQHKSKKKPSVVITWMSLDFYTKVLKSCRSLLESVQKLDLEATIDKVVKIYDALIYMQVNSSFEDHILEDLC | 591 |
| 592 | GMLSLPWIYSHSDDGCLKLTTFAANLLTLSCRISDSYSPQAQSRCVFLLTLFPRRIFLEWRTAVYNWALQSSHEV | 666 |
| 667 | IRASCVSGFFILLQQQNSCNRVPKILIDKVKDDSDIVKKEFASILGQLVCTLHGMFYLTSSLTEPFSEHGHVDLF | 741 |
| 742 | CRNLKATSQHECSSSQLKASVCKPFLFLLKKKIPSPVKLAFIDNLHHLCKHLDFREDETDVKAVLGTLLNLMEDP | 816 |
| 817 | DKDVRVAFSGNIKHILESLDSEDGFIKELFVLRMKEAYTHAQISRNNELKDTLILTTGDIGRAAKGDLVPFALLH | 891 |
| 892 | LLHCLLSKSASVSGAAYTEIRALVAAKSVKLQSFFSQYKKPICQFLVESLHSSQMTALPNTPCQNADVRKQDVAH | 966 |
| 967 | QREMALNTLSEIANVFDFPDKNRFLTRTLQVLLPDLAAKASPAASALIRTLGKQLNVNREEILENNFKYIFSHLV | 1041 |
| 1042 | CSCSKDELERALHYLKNETEIELGSLLRQDFQSLHNELLLRIGEHYQQVFNGLSILASFASSDDPYQGPRDIISP | 1116 |
| 1117 | ELMADYLQPKLLGILAFFNMQLLSSSVGIEDKKMALNSLMSLMKLMGPKHVSSVRVKMMTTLRTGLRFKDDFPEL | 1191 |
| 1192 | CCRAWDCFVRCLDHACLGSLLSHVIVALLPLIHIQPKETAAIFHYLIIENRDAVQQFLHEIYFLPDHPELKKIKA | 1266 |
| 1267 | VLQEYRKETSESTDLQTTLQLSMKAIQHENVDBRIHALTSLKETLYKNQEKLIKYATDSETVEPIISQLVTVLLK | 1341 |
| 1342 | GCQDANSQARLLCGECLGELGAIDPGRLDFSTTETQGKDFTFVTGVEDSSFAYGLLMELTRAYLAYADNSRAQDS | 1416 |
| 1417 | AAYAIQELLSIYDCREMETNGPGHQLWRRFPEHVREILIPHLNTRYKSSQKSTDWSGVKKPIYLSKLGSNFAEWS | 1491 |
| 1492 | ASWAGYLITKVRHDLASKIFTCCSIMMKHDFKVIIYLLPHILVYVLLCCNQEDQQEVYAEIMAVLKHDDQHTINT | 1566 |
| 1567 | QDIASDLCQLSTQTVFSMLDHLTQWARHKFQALKAEKCPHSKSNRNKVDSMVSTVDYEDYQSVTRFKDKIPQDTL | 1641 |
| 1642 | AVASFRSKAYTRAVMHFESFITEKKQNIQEHLGFLQKLYAAMHEPDGVAGVSAIRKAEPSLKEQILEHESLGLLR | 1716 |
| 1717 | DATACYDRAIQLEPDQIIHYHGVVKSMLGLGQLSTVITQVNGVHANRSEWTDELNTYRVEAAWKLSQWDLVENYL | 1791 |
| 1792 | AADGKSTTWSVRLGQLLLSAKKRDITAFYDSLKLVRAEQIVPLSAASFERGSYQRGYEYIVRLHMLCELEHSIKP | 1866 |
| 1867 | LFQHSPGDSSQEDSLNWVARLEMTQNSYRAKEPILALRRALLSLNKRPDYNEMVGECWLQSARVARKAGHHQTAY | 1941 |
| 1942 | NALLNAGESRLAELYVERAKWLWSKGDVHQALIVLOKGVELCFPENETPPEGKNMLIHGRAMLLVGRFMEETANF | 1016 |
| 2017 | ESNAIMKKYKDVTACLPEWEDGHFYLAKYYDKLMPMVTDNKMEKQGDLIRUIVLHFGRSLQYGNQFIYQSMPRML | 2091 |
| 2092 | TLWLDYGTKAYEWEKAGRSDRVQMRNDLGKINKVITEHTNYLAPYQFLTAFSQLISRICHSHDEVFVVLMEIIAK | 2166 |
| 2167 | VFLAYPQQAMWMMTAVSKSSYPMRVNRCKEILNKAIHMKKSLEKFVGDATRLTDKLLELCNKPVDGSSSTLSMST | 2241 |
| 2242 | HFKMLKKLVEEATFSEILIPLQSVMIPTLPSILGTHANHASHEPFPGHWAYIAGFDDMVEILASLQKPKKISLKG | 2316 |
| 2317 | SDGKFYIMMCKPKDDLRKDCRLMEFNSLINKCLRKDAESRRRELHIRTYAVIPLNDECGIIEWVNNTAGLRPILI | 2391 |
| 2392 | KLYKEKGVYMTGKELRQCMLPKSAALSEKLKVFREFLLPRHPPIFHEWFLRTFPDPTSWYSSRSAYCRSTAVMSM | 2466 |
| 2467 | VGYILGLGDRHGENILFDSLTGECVHVDFNCLFNKGETFEVPEIVPFRLTHNMVNGMGPHGTEGLFRRACEVTMR | 2541 |
| 2642 | LMRDQREPLMSVLKTFLHDPLVEWSKPVKGHSKAPLNETGEVVNEKAKTHVLDIEQRLQGVIKTRNRVTGLPLSI | 2616 |
| 2617 | EGHVHYLIQEATDENLLCQMYLGWTPYM | 2664 |
| Sequence ID No. 3: rad3.seq | | |
| 1 | GGTACCAAGTAAAAACTGCTTAGTAAGTATAAAACACAGAAGAATCCGCGCTCTAGTGAACCAATGCCCTGCGTA | 75 |
| 76 | TGACGCTCCACTGACGCTATAGTCAATGAGAACTAGGATGTGCGATTATAACTTATCTTTTCAATATTTTCTTAT | 150 |
| 151 | TATTTATTTAAGAAATAATTGAATTAAAACTCATTTCTTCTTTTATTAGCCGTAAAATAGCTTATTTTCTCTCCT | 225 |
| 226 | ACTACCTTTCAACAATAACTTTTTTTTTTCTTTATTCACCATTATAATCACATCAAAAGTCAAAAAATTCAATCA | 300 |
| 301 | TTATCAGAAACATCCAGCCTAATATTACTTAAAAGTTAGTTTCCTCTGAAAATTCAGTATCACAAAAGCTCGTTA | 315 |
| 376 | ATTAGCATCGCTCGATACTTAGTGCACCATGCATCTTCCTTTACCTCGTGAGTGGAAATCGATTTGATAATCGAT | 450 |
| 451 | TGCCACTTTTCGCATAATTCTATTGAGATATTTTATTACTTACAATCGTCTTTTATAAATGCTCAAGACTTTGAA | 525 |
| 526 | CGCGCGTGTTGCGTTTTAAAAAGGCCTTTTTTTGAATTGAATCAATGGTTTGATATAGTATGAGCCAACACGCAA | 600 |
| 601 | AAAGGAAAGCTGGGTCACTCGATCTTTCACCCAGAGGCTTAGATGACAGACAGGCTTTCGGACAGCTTTTCAAAG | 675 |
| 676 | AAGTATTAGCATTAGACAAAGAACATGAGTTAGGTAGAAGTAATTCTTTACCATCTATGACCTCCGAGCTTGTTG | 750 |
| 751 | AAGTTTTAATTGAAGTTGGTCTTCTAGCTTTTAAACATGATGATTCAAAATCTGAATTTATCTCTCCTAAGATGC | 825 |
| 826 | TAAAAGAAGCCCATCTCTCTCTACAAGCGTTAATGCTAATCTTAAAAAGGTCTCCGACAGTTTTGCGGGAGATTA | 900 |
| 901 | AATCATCTGTTACTCTTTTGGATTGGATTTTACCCAGGACTATATCATTGTTTGCTGATATTCGTTTTATTAAGT | 975 |
| 976 | TATTTGACTCATTAAAAGAGTTTCATAAGCTAATTTATCAGCTAATCAGTGAAAAGTCATTCCTATGGGACTTAT | 1050 |
| 1051 | ATGCTTCGTTTATGCGTTATTGGAAATATTATATTACAAACGTTTCTTCTATCGTTCTCCAAATCACTAATGCTA | 1125 |
| 1126 | CATTCCCTTACAAGATGCCCTCACCCAATTCTCAACCATTGCAGAGTATCTCCCCAAATTATCCAACCCATCGAG | 1200 |
| 1201 | AGGACAAATTTGATTTACTTATCATTAATATAGAGGAGGCTTGTACATTTTTCTTTGAAAGTGCCCATTTTTTTG | 1275 |
| 1276 | CACAATGCTCATATTTAAAGAAATCCAATTTTCCTAGTCCACCTCTCTTTACAGCGTGGACTTGGATCAAGCCAT | 1350 |
| 1351 | GTTTTTTTAATTTTGTTATTTTATTAAAACGAATCAGCATCGGAGACTCACAGCTCTTTCTACATTTGCATTCAC | 1425 |
| 1426 | GTATAGTCCAAACTTTATGCTGTTTTTCCTTGAATTTTATATATCATGGCCTTCCCATTTGTGAAAAATCTAAAC | 1500 |
| 1501 | ATATTTTAATGTCCTCCATCAACTTAACATTGGGATCATTGAAGAAAACTTATACAGTTGCTAATACTGCTATAT | 1575 |
| 1576 | CTCTTTTTTTTCTCTCTTTATTTCTTTTTACCCAAAACTGTAGCTGGTCATTCTATCCTTTTGGGGTTTCCTTAC | 1650 |
| 1651 | TTTCTGACTTCAAGGTATTAGAGCAACTTGAACCAGATTCTGATCTCAAAAAGGCAATAATATTATTTAAGTGCA | 1725 |
| 1726 | GATACCAAAGTTCAGAAATAGATCAAACAACTCTCCGTGCTTTTGGCGAAATTTGTACTGGTAAACTTGAAAACA | 1800 |
| 1801 | CGTTCTTTTCTAACTCTGAATTAAACCTTTTTCTTTTACATTATCTTTCCTTGGACAATGACTTGTCAAATATTC | 1875 |
| 1876 | TTAAAGTGGATTTCCAGAATGGTCATAACATATGTACATTTGCAAAATGGTGTATAAACAACAACTTAGATGAAC | 1950 |
| 1951 | CGTCTAATTTAAAGCACTTTCGTGAAATGTTAGATTATTATAGCTCTCATAATGTTACAATAAGTGAGGACGACC | 2025 |
| 2026 | TGAAGAACTTCTCTTTAGTTTTGTGTACTCATGTTGCAAAGGTGAATGAGAAAACAAATAGTATTTTCCGCACAT | 2100 |
| 2101 | ATGAAGTACATGGTTGTGAAGTTTGTAACTCATTTTGTTTACTATTTGATGAGCGGTCGCCTTTTAAAATTCCTT | 2175 |
| 2176 | ATCACGAATTGTTTTGTGCATTGCTAAAAAATCCCGACATAATTTCCTCTTCTGTTAAACAATCATTGTTGCTTG | 2250 |
| 2251 | ATGGCTTTTTTCGGTGGAGCCAGCATTGCTCAAACTTTAATAAAGAATCAATGTTAAGTTTAAGAGAATTTATTA | 2325 |
| 2326 | TGAAAGCATTAGCCAGTACTTCAAGATGTTTACGTGTTGTTGCTGCAAAAGTTTTGCCCATTTTCATTAAGGGAC | 2400 |
| 2401 | CTAATAATCTTGATATAGTTGAATTTCACAAGGAAAGTAAAGCCTTGATTTTTAATACGTTGAAAATATTGGCGG | 2475 |
| 2476 | TGGAAAATACAGCTATTTTAGAAACGGTCATTCTTTCCTGGATCTCCTTATCTAGAGTGGTAGAAGAAGAAGAAT | 2550 |
| 2551 | TACATTTTGTACTATTGGAAGTTATATCTTCTGTGATAAACAGCGGAATATTTTATCAAGGCATTGGTCTCAGCG | 2625 |
| 2626 | CTCTGCAACAAATTGCCTCGACGCGTCATATATCCGTTTGGCAATTACTTTCTCCATATTGGCCAACAGTGTCCG | 2700 |
| 2701 | TTGCGATTGTCCAAGGTATGGGTAAAAAACCGAACATAGCCAGTTTATTTGCTCAGCTTATGAATATTTCCGAGG | 2775 |
| 2776 | GCGATTTTCTTATTCGAACACAGGCGTACACTTTACCATTCCTTGTACTTACTAAAAACAAAGCGTTAATAGTAC | 2850 |
| 2851 | GTATAGCTGAACTTTCACAAAGTGATGTTGCTACTTTGTGCCTTACCAATATGCATAAAATCCTTGCTTCGCTAC | 2925 |
| 2926 | TTACTACGGATCATCCTAATTTGGAAGAGAGTGTGATGCTTCTTCTTTCACTGGCCACTTCTGATTTTGAAAAAG | 3000 |
| 3001 | TTGATTTAACGTCTTTGTTACGCTCTGATCCTATTTCTATTACTGTGGAGTTGTTACAGCTTTATCAGAATGATG | 3075 |
| 3076 | TTCCTCATGAAAAAATTGAAAATGCTTTAAGAAAGGTAGCAATGATTGTCTCTCAAGTGGTTAATGACGAAGACT | 3150 |
| 3151 | TGAGCAATAAGGAATTACTTTATGATTTTTTTAATAATCACATTTTGGGTATCTTAGCAGAATTTTCTAATATCC | 3225 |
| 3226 | TTAACGACCTGAAAGGAAAGACTTCAATTAATGAAAAGATTAAGACAATTGTCGGCATTGAAAAAATGTTATCTT | 3300 |
| 3301 | TATGTGGAGGTGCAGTCAAACTTGGATTACCACAGATACTTTCTAATTTACAAAGTGCTTTTCAAAATGAGCACT | 3375 |
| 3376 | TAAGGTTTTATGCAATCAAAGCTTGGTTCAGTTTGATATTAGCAACCAAGGAGCCCGAGTATAGTTCAATTGCTG | 3450 |
| 3452 | GTTTAAGTCTTGTAATTTTACCTCCTTTATTCCCTTATTTAGAACCACAAGAAGCAGAGCTAGTAATTCAAATAT | 3525 |
| 3526 | TTGATTTTATTTCTTCTGACACACACAAGTGCCTACAAGGATTAAAGTGGGCTATCCCCACCAGTCTGGATTCAG | 3600 |
| 3601 | CGTGCTTTAGCCTTAAGGCTAAAGAAATATTCTGTTCGCTTCAAAATGAAGATTTTTACTCTGAGCTTCAAAGTA | 3575 |
| 3676 | TAATTAAGTGTTTAACTAACGAAAATGAGCCAGTTTGTTATTTAGGTTTACAAAAATTAGAACTTTTTTTTCAAG | 3750 |
| 3751 | CCAAGGTGGACGAGTTACATGACACACTAAATTTGGACATATCCAACGAAGTTCTGGACCAATTACTAAGATGCC | 3825 |
| 3826 | TTTTAGATTGTTGTGTAAAATATGCTTCAACAAATATGCAAATATCATATCTTGCTGCAAAAAATCTTGGTGAAT | 3900 |
| 3901 | TGGGTGCGATAGATCCCAGCCGCGCCAAGGCTCAACATATTATTAAAGAAACAGTTGTTCTTGATAACTTTGAAA | 3975 |
| 3976 | ACGGAGAAGAAAGTTTGAAGTTTATTCTAGATTTTATGCAATCGCAGTTAATTCCAGCTTTCCTTGTTACTACTG | 4050 |
| 4051 | ATACTAAAGCACAAGGTTTTCTTGCCTATGCTCTGCAAGAGTTTCTAAAGCTTGGTGGATTCAAGTCCGCAGTGA | 4125 |
| 4126 | TTAATAAAAAAAAGGGACTAACTGTGGTAACAGAACATTGGATGTCTTTGCCTGATTTATCCAAACGTGTGCTTA | 4200 |
| 4201 | TACCATTTTTAACTTCCAAGTATCATTTAACACCAATCCCCAAAATTGACATTCGGTACCCTATTTATAAAGAAA | 4275 |
| 4276 | ATGTTACTATTCATACTTGGATGCACTTGTTTTCTCTTAAATTGATGGAGTACGCCCATTCGCAAAACGCTGAAA | 4350 |
| 4351 | AAATATTTGGTATTTGTTCGAAAGTAGTGAAAGACCAAGAGGTTAACATTCCCTGTTTTCTTCTTCCCTTTCTTG | 4425 |
| 4426 | TTTTAAATGTTATTTTAACCGAGTCAGAACTGGAAGTTAATAAAGTCATTGAAGAATTCCAGCTTGTTATTAATC | 4500 |
| 4501 | AACCGGGACCTGATGGATTAAATTCCGTGGGGCAACAAAGATACACCTCATTTGTAGATGTATTTTTTAAGATTG | 4575 |
| 4576 | TGGATTACCTTAACAAATGGCTTCGCATGCGAAAGAAGAGGAATTGGGATAGACGTTCTGCCATTGCAAGGAAAG | 4650 |
| 4651 | AGAACCGTTATATGTCGGTGGAAGATGCTACCTCTCGAGAATCATCGATCTCAAAAGTTGAGTCATTTCTTTCTC | 4725 |
| 4726 | GATTTCCTTCAAAAACATTAGGTATTGTCTCTTTAAATTGTGGATTTCATGCTCGTGCATTGTTTTATTGGGAGC | 4800 |
| 4801 | AACACATACGTAATGCTACAGCTCCATATGCAGCTTTAGAGTCCGATTATAGAGTTTTGCAGGAAATATATGCTG | 4875 |
| 4876 | GAATTGATGATCCAGATGAAATCGAAGCAGTGTCTTTAAATTTCCATGATTACTCGTTTGATCAACAACTCCTTT | 4950 |
| 4951 | TACATGAAAATTCAGGAACATGGGACTCGGCTTTGAGTTGTTACGAAATTATTATTCAAAAGGATCCTGAAAATA | 5025 |
| 5026 | AAAAGGCGAAAATCGGTTTGCTTAACAGCATGCTGCAATCGGGGCATTATGAATCTCTTGTTTTGAGTTTAGATT | 5100 |
| 5201 | CTTTTATAATCAATGACAACCACGAGTATTCGAAGATGTTAAATTTGGGTATTGAAGCTTCATGGCGTTCGCTAT | 5175 |
| 5176 | CTATTGATTCGTTAAAAAAGTGTCTTTCAAAAAGCAACTTGGAATCTTTCGAAGCTAAATTGGGTAGCATATTTT | 5250 |
| 5251 | ACCAATACCTACGGAAGGATTCTTTTGCTGAATTGACGGAGCGGCTGCAACCCTTGTACGTTGATGCTGCTACAG | 5325 |
| 5326 | CAATTGCAAACACAGGCGCCCATTCAGCCTATGATTGTTATGATATTTTATCTAAGCTGCACGCAATTAATGACT | 5400 |
| 5401 | TTAGTAGGATTGCTGAAACTGACGGAATTGTTTCCGACAATCTTGATATTGTTCTTCGCCGTCGGCTTAGCCAAG | 5475 |
| 5476 | TAGCTCCGTACGGTAAATTCAAGCACCAAATCCTGTCCACTCACTTAGTTGGCTATGAAAAATTTGAAAACACGA | 5550 |
| 5551 | AGAAAACTGCTGAAATATATCTCGAGATTGCAAGAATATCTCGAAAAAATGGTCAATTTCAAAGAGCCTTCAATG | 5625 |
| 5526 | CCATCCTCAAAGCAATGGATTTAGATAAACCGCTAGCAACAATAGAGCACGCACAATGGTGGTGGCATCAAGGGC | 5700 |
| 5701 | AACATCGTAAAGCTATTTCTGAATTGAATTTTTCGCTTAATAACAACATGTTTGATTTGGTTGATGAGCATGAAG | 5775 |
| 5776 | AAAGACCTAAAAATCGTAAAGAAACTTTAGGAAATCCACTTAAAGGAAAAGTGTTCTTGAAACTTACAAAATGGC | 5850 |
| 5851 | TCGGAAAAGCTGGCCAACTGGGATTGAAGGATTTGGAGACGTATTATCATAAAGCGGTAGAGATTTACTCAGAAT | 5925 |
| 5925 | GTGAGAATACGCATTATTATCTTGGCCATCATCGAGTTTTAATGTATGAAGAAGAACAAAAGCTCCCAGTTAATG | 6000 |
| 6001 | AACAGAGCGAACGATTTTTAAGTGGTGAGTTAGTAACTCGCATAATTAACGAATTTGGTCGATCTTTGTACTATG | 6075 |
| 6076 | GTACAAATCATATATATGAAAGTATGCCAAAATTGCTCACACTGTGGCTTGATTTTGGGGCCGAAGAACTTCGCT | 6150 |
| 6251 | TATCTAAAGATGACGGCGAAAAGTACTTTCGTGAACACATTATCTCTTCGAGAAAAAAATCTTTGGAACTTATGA | 6225 |
| 6226 | ATTCGAATGTTTGTCGCCTTTCTATGAAAATTCCTCAATACTTTTTTCTGGTTGCATTATCCCAAATGATATCCA | 6300 |
| 6301 | GAGTATGCCATCCAAATAATAAAGTTTATAAAATTTTGGAACATATAATTGCAAACGTTGTAGCATCTTATCCTG | 6375 |
| 6376 | GGGAGACGCTATGGCAATTAATGGCAACAATAAAATCGACTTCTCAAAAGCGCTCGCTTCGTGGAAAAAGCATTT | 6450 |
| 6451 | TAAATGTTTTACATTCTAGGAAGCTTTCTATGTCTTCCAAAGTTGATATAAAAGCACTCAGTCAATCTGCAATTC | 6525 |
| 6526 | TCATTACTGAAAAGTTAATCAATTTGTGCAATACAAGGATTAACAGTAAATCTGTAAAAATGAGCTTAAAGGATC | 6600 |
| 6601 | ATTTTCGGCTTTCTTTTGATGATCCGGTAGATTTAGTCATTCCTGCTAAATCATTTTTAGACATTACTTTACCAG | 6675 |
| 6676 | CTAAAGATGCTAACAGAGCTAGTCATTATCCATTTCCAAAAACTCAGCCTACTCTGTTGAAATTTGAGGATGAGG | 6750 |
| 6751 | TGGATATAATGAACTCTCTTCAAAAACCAAGAAAAGTGTACGTTAGAGGTACGGATGGCAACTTATACCCATTCT | 6325 |
| 6826 | TGTGCAAACCCAAAGATGATCTTCGTAAGGATGCTAGATTGATGGAATTTAATAATCTTATTTGTAAAATATTGA | 6900 |
| 6901 | GGAAAGATCAAGAAGCGAACAGAAGGAACTTGTGTATTAGAACTTATGTTGTTATTCCTTTAAATGAAGAATGCG | 6975 |
| 6976 | GATTTATCGAATGGGTAAATCATACTCGTCCATTTAGAGAAATTTTGTTAAAAAGCTATAGACAGAAAAACATTC | 7050 |
| 7051 | CCATATCATATCAAGAAATCAAAGTTGATTTAGACTTTGCACTGCGAAGTCCTAACCCTGGTGATATATTTGAAA | 7125 |
| 7126 | AGAAAATCTTACCGAAATTTCCTCCAGTTTTTTATGAGTGGTTTGTTGAATCTTTCCCAGAACCAAATAATTGGG | 7200 |
| 7201 | TTACTAGTAGACAAAACTATTGCCGAACTTTAGCAGTAATGTCAATAGTTGGCTACGTTTTGGGTTTGGGAGATC | 7275 |
| 7276 | GCCATGGCGAAAACATATTGTTTGATGAATTTACAGGTGAAGCTATCCATGTCGATTTCAACTGTCTTTTTGATA | 7350 |
| 7351 | AAGGTCTTACTTTTGAAAAACCTGAAAAGGTGCCGTTCAGATTAACTCATAATATGGTAGATGCAATGGGTCCGA | 7425 |
| 7426 | CAGGTTATGAAGGGGGTTTCAGGAAAGCTAGCGAAATAACGATGCGGCTTCTTCGCTCAAACCAAGATACATTGA | 7500 |
| 7501 | TGAGCGTACTAGAGTCTTTCCTACATGATCCTTTAGTCGAGTGGAATAGAAAGAAGTCGTCAAGCAAGTACCCGA | 7575 |
| 7576 | ATAATGAAGCAAATGAAGTTTTTGGATATAATTCGCAAAAATTTCAAGGCTTTATGCCAGGGGAGACGATACCTT | 7650 |
| 7651 | TATCTATTGAAGGGCAAATTCAAGAATTGATCAAATCTGCTGTCAACCCAAAAAACCTGGTAGAAATGTACATTG | 7725 |
| 7726 | GTTGGGCTGCTTATTTCTAGCATTTTACTAACAAAAATTTCAATGAACAAGCTACCCATTATTAAACTTATGATT | 7800 |
| 7801 | TGAATCGAAGATATTTTATTTATTAATCCGATGAAGAATTCTCGCTGAGTTGTTCAATTTCTTGTAATTTTCCTT | 7875 |
| 7876 | CCATTTCTAAATCGTCGATTCGCTTAAATAGGGCACTGGCTTTTTGTGCATTTTTCTCTCGTAAAGCAGCTTCTG | 7950 |
| 7951 | ATTGAAAAAAAGCTATATCTGTTTCTGAGTCATCATCCGAATCAACAATATATTTTGCAGATCGACCTGCAG | 8022 |
| Sequence ID No. 4: rad3 protein | | |
| 1 | MSQHAKRKAGSLDLSPRGLDDRQAFGQLLKEVLALDKEHELGRSNSLPSMTSELVEVLIEVGLLAFKHDDSKSEF | 75 |
| 76 | ISPKMLKEAHLSLQALMLILKRSPTVLREIKSSVTLLDWILPRTISLFADIRFIKLFDSLKEFHKLIYQLISEKS | 150 |
| 151 | FLDLYASFMRYWKYYITNVSSIVLQITNATFPYKIIPSPNSQPLQSISPNYPTHREDKVDLLIINIEEACTFFFE | 225 |
| 226 | SAHFFAQCSYLKKSNFPSPPLFTAWTWIKPCFFNFVILLKRISIGDSQLFLHLHSRIVQTLCCFSLNFIYHGLPI | 300 |
| 301 | CEKSKHILMSSINLTLGSLKKTYTVANTAISLFFLSLFVLPKTVAGLFYPFGVSLLSDFKVLEQLEPDSDLKKAI | 375 |
| 376 | ILFKCRYQSSEIDQTTLRAFGEICTGKLENTLFSNSELNLFLLHYLSLDNDLSNILKVDFQNGHNICTFAKWCIN | 450 |
| 451 | NNLDEPSNLKHVREMLDYYSSKNVTISEDDLKNFSLVLCTHVAKVNEKTNSIFRIYEVHGCEVCNSFCLLFDERS | 525 |
| 526 | PFKIPYHELFCALLKNPDIISSSVKQSLLLDGFFRMSQHCSNFNKESMLSLREFIMKALASTSRCLRVVAAKVLP | 600 |
| 601 | IFIKGPNNLDIVEFHKESKALIFNTLKILAVENTAILETVILSWISLSRVVEEEELHFVLLEVISSVINSGIFYQ | 675 |
| 676 | GIGLSALQQIASTRHISVWQLLSPYWPTVSVAIVQGMGKKPNIASLFAQLMNISEGSFLIRTQAYTLPFLVLTKN | 750 |
| 751 | KALIVRIAELSQSDVATLCLTNMHKILASLLTTDHPNLEESVMLLLSLATSDFEKVDLTSLLRSDPISITVELLQ | 825 |
| 826 | LYQNDVPHEKIENALRKVAMIVSQVVNDEDLSNKELLYDFFNMHILGILAEFSNILNDLKGKTSINEKIKTIVGI | 900 |
| 901 | EKMLSLCGGAVKLGLPQILSNLQSATQNEHLRFYAIKAWFSLILATKEPEYSSIAGLSLVILPPLFPYLEPQEAE | 975 |
| 976 | LVIQIFDFISSDTHKCLQGLKWAIPTSLDSACFSLKAKEIFCSLQNEDFYSELQSIIKCLTNENEPVCYLGLQKL | 1050 |
| 1051 | ELFFQAKVDELHDTLNLDISNEVLDQLLRCLLDCCVKYASTNMQISYLAAKNLGELGAIDPSTAKAQHIIKETVV | 1125 |
| 1126 | LDNFENGEESLKFILDFMQSQLIPAFLVTTDTKAQGFLAYALQEFLKLGGFKSAVINKKKGLTVYTEHWMSLPDL | 1200 |
| 1201 | SKRVLIPFLTSKYHLTPIPKIDIRYPIYKENVTIHTWMQLFSLKLMEYAHSQNAEKIFGIVSKVVKDQEVNIPCF | 1275 |
| 1276 | LLPFLVLNVILTESELEVNKVIEEFQLVINQPGPDGLNSVGQQRYTSFVDVFFKIVDYLNKWLRMRKKRVWSRRS | 1350 |
| 1352 | AIARKENRYMSVEDATSRESSISKVESFLSRFPSKTLGIVSLNCGFHARALFYWEQHIRNATAPYAALESDYRVL | 1425 |
| 1426 | QEIYAGIDDPDEIEAVSLNFHDYSFDQQLLLHENSHTWDSALSCYEIIIQKDPENKKAKIGLLNSMLQSGHYESL | 1500 |
| 1501 | VLSLDSFIINDNHEYSKMLNLGIEASWRSLSIDSLKKCLSKSNLESFEAKLGSIFYQYLRKDSFAELTERLQPLY | 1575 |
| 1576 | VDAATAIANTGAHSAYDCYDILSKLHAINDFSRIAETDGIVSDNLDINLRRRLSQVAPYGKFKHQILSTHLVGYE | 1650 |
| 1651 | KFENTKKTAEIYLEIARISRKNGQGQRAFNAILKAMDLDKPLATIEHAQWWWHQGQHRKAISELNFSLNNNMFDL | 1725 |
| 1726 | VDEHEERPKNRKETLGNPLKGKVFLKLTKWLGKAGQLGLKDLETYYHKAVEIYSECENTHYYLGHHRVLMYEEEQ | 1800 |
| 1801 | KLPVNEQSERFLSGEKVTRIINEFGRSLYYGTNHIYESMPKLLTLWLDFGAEELRLSKDDGEKYFREHIISSRKK | 1875 |
| 1876 | SLELMNSNVCRLSHKIPQYFFLVALSQMISRVCHPNNKVYKILEHIIANVVASYPGETLWQLMATIKSTSQKRSL | 1950 |
| 1951 | RGKSILHVLHSRKLSMSSKVDIKALSQSAILITEKLINLCNTRINSKSVKMSLKDHFRLSFDDPVDLVIPAKSFL | 2025 |
| 2026 | DITLPAKDANRASHYPFPKTQPTLLKFEDEVDIHNSLQKPRKVYVRGTDGNLYPFLCKPKDDLRKDARLMEFNNL | 2100 |
| 2101 | ICKILRKDQEANRRNLCIRTYVVIPLNEECGFIEWVNHTRPFREILLKSYRQKNIPISYQEIKVDLDFALRSPNP | 2175 |
| 2176 | GDIFEKKILPKFPPVFYEWVVESVPEPNNWVTSRQNYCRTLAVMSIVGYVLGLGDRHGENILFDEFTGEAIHVDF | 2250 |
| 2251 | NCLFDKGLTVEKPEKVPFRLTHNNVDAMGPTGYEGGFRKASEITMRLLRSNQDTLNSVLESFLHDPLVEWNRKKS | 2325 |
| 2326 | SSKYPNNEANEVLQIIRKKFQGFMPGETIPLSIEGQIQELIKSAVNPKNLVEMYIGWAAYF | 2386 |

In italics. sequenced by Seaton et al.
In Bold are those bases deleted in Seaton et al. (2499. 22501. 2507. 2509)
Underlined are the two bases either side of a single C insert (5918/5919) in
Seaton et 81. (i.e. the incorrect base not shown, but the one residue either side is)

1.-17. (canceled)

18. A purified and isolated ATR polypeptide that has lipid kinase activity, said polypeptide encoded by a polynucleotide selected from the group consisting of:
(a) a polynucleotide encoding the amino acid sequence set out in SEQ ID NO: 2,
(b) a polynucleotide encoding the amino acid sequence set out in SEQ ID NO: 4, and
(c) a polynucleotide which hybridizes to the complement of the polynucleotide of (a) or (b) under conditions including a final wash at 55° C.

19. A purified and isolated ATR polypeptide that has lipid kinase activity, said polypeptide encoded by a polynucleotide selected from the group consisting of:
a) a polynucleotide set out in SEQ ID NO: 1;
b) a polynucleotide set out in SEQ ID NO: 3; and
c) a polynucleotide that hybridizes to the complement of the polynucleotide of (a) or (b) under conditions including a final wash at 55° C.
